(19) United States
(12) Patent Application Publication
(10) Pub. No.: US 2022/0142967 A1
(43) Pub. Date: May 12, 2022

(54) Methods and Compositions for the Treatment of Multiple Sclerosis

(71) Applicant: SYMBIONYX PHARMACEUTICALS INC, Boonton, NJ (US)

(72) Inventors: Ratan K. Chaudhuri, Lincoln Park, NJ (US); Krzysztof Bojanowski, Santa Paula, CA (US)

(21) Appl. No.: 17/566,804

(22) Filed: Dec. 31, 2021

Related U.S. Application Data
(63) Continuation-in-part of application No. 16/400,360, filed on May 1, 2019, now abandoned.
(60) Provisional application No. 62/665,890, filed on May 2, 2018.

(51) Int. Cl. A61K 31/34

(52) U.S. Cl. CPC A61K 31/34

(57) ABSTRACT

Compositions and methods based on isosorbide esters for the treatment of multiple sclerosis.

### RELATED APPLICATIONS

This application is a Continuation-in-Part of pending U.S. patent application Ser. No. 16/400,360, filed May 1, 2019, which claims the benefit of prior U.S. Provisional Patent Application No. 62/665,890, filed on May 2, 2018, the entire contents of both of which are hereby incorporated by reference.

### TECHNICAL FIELD

The present teaching is directed to compositions and methods of using isosorbide di-(methyl fumarate) in the treatment of multiple sclerosis.

### BACKGROUND

Multiple sclerosis (MS) is a chronic autoimmune disease of the Central Nervous System (CNS), estimated to affect over 2.1 million people worldwide with more than 400,000 cases in the United States alone. The knowledge of this disease is vast; though still incomplete and lacking as evident from http://www.nationalmsscocietyorg/about-multiple-sclerosis/what-we-know-aboutms/fags-about-ms/indexaspx: National Multiple Sclerosis Society. The symptoms vary and include motor deficits such as ataxia and spasticity, vision disturbances, bladder and rectal disorders, fatigue, and others (T Menge et al., Disease-modifying agents for multiple sclerosis: recent advances and future prospects, Drugs, 68(17):2445-68, 2008. doi: 10.2165/0003495-200868170-00004). MS is the second most common cause of disability in young adults, after trauma.

Currently, only six medications are approved for immunomodulatory and immunosuppressive treatment of the relapsing disease course and secondary-progressive MS (T Menge et al., Disease-modifying agents for multiple sclerosis: recent advances and future prospects, Drugs, 68(17):2445-68, 2008. doi: 10.2165/0003495-200868170-00004). During the last decade, understanding of autoimmunity and the pathogenesis of MS has advanced substantially. This has led to the development of a number of compounds, several of which are currently undergoing clinical testing in phase II and III studies. While current treatment options are only available for parenteral administration, several oral compounds are now in clinical trials, including the immunosuppressive agents—cladribine and laquinimod. A novel mode of action has been described for fingolimod, another orally available agent, which inhibits egress of activated lymphocytes from draining lymph nodes. Dimethyl fumarate (DMF) has been shown to exhibit immunomodulatory as well as immunosuppressive activity when given orally. All of these compounds have successfully shown efficacy, at least in regards to the surrogate marker contrast-enhancing lesions on magnetic resonance imaging.

Another class of agents that is highlighted are biological agents, namely monoclonal antibodies (mAb) and recombinant fusion proteins. The humanized mAb daclizumab inhibits T-lymphocyte activation via blockade of the interleukin-2 receptor. Alemtuzumab and rituximab deplete leukocytes and B cells, respectively; the fusion protein atacicept inhibits specific B-cell growth factors resulting in reductions in B-cells and plasma cells. These compounds are currently being tested in phase II and III studies in patients with relapsing MS. The concept of neuro-protection and -regeneration has not advanced to a level where specific compounds have entered clinical testing. However, several agents approved for conditions other than MS are highlighted. Finally, with the advent of these highly potent novel therapies, rare, but potentially serious adverse effects have been noted, namely infections and malignancies (T Menge et al., Disease-modifying agents for multiple sclerosis: recent advances and future prospects, Drugs, 68(17):2445-68, 2008. doi: 10.2165/0003495-200868170-00004).

MS is triggered by autoreactive T cells against myelin antigens. Similar to psoriasis vulgaris, MS is mainly a Th1-cell disorder. This may be a very important part of the effect of Dimethyl Fumarate (DMF) on MS. Yet up until now, no Th1/Th2 shift, but rather a significant reduction in microglial cells and macrophages, has been observed.

Scientific investigations are still in progress to clarify the ultimate mechanism of action responsible of the treatment effects of DMF. What has become clear thus far is that, similar to other medications such as interferon, DMF does not have a single mechanism of action but rather has a multitude of biological effects (R Bomprezi, Dimethyl fumarate in the treatment of relapsing-remitting multiple sclerosis: an overview, Therapeutic Avd Neurol Disorder, 8(1):20-30, 2015; references cited therein). In vitro studies have revealed that DMF has anti-inflammatory properties linked to its ability to promote a Th2 immune response. Added to cultures of stimulated peripheral mononuclear blood cells, Monomethyl fumarate (MMF) enhanced the production of interleukin-4 (IL-4) and IL-5, cytokines characteristic of the Th2 phenotype, in a dose-dependent fashion (de Jong et al., Selective stimulation of T helper 2 cytokine responses by the anti-psoriasis agent MMF, Eur J Immunol 26: 2067-2074, 1996). Furthermore, besides affecting T lymphocytes, a shift to a Th2 profile was confirmed and replicated in dendritic cells (Litjens et al., Monomethylfumarate affects polarization of monocyte-derived dendritic cells resulting in down-regulated Th1 lymphocyte responses. Eur J Immunol 34: 565-575, 2004; Litjens et al., Effects of monomethylfumarate on dendritic cell differentiation. Br J Dermatol 154: 211-217, 2006), and it is fair to say that directing the immune response away from Th1 is a likely mechanism by which DMF exerts some of its immuno-modulatory effects.

Additional in vivo and in vitro experiments have further clarified the impact of DMF on type II dendritic cells, providing more details on the cascades of events that follow exposure to DMF (Ghoreschi et al. Fumarates improve psoriasis and multiple sclerosis by inducing type II dendritic cells. J Exp Med 208: 2291-2303, 2011). In the end, the impact on T lymphocytes seems to be just a portion of the modifications induced by DMF, which influences several other cells, including macrophages, microglia, astrocytes and neurons (Moharregh-Khiabani et al. Fumaric acid and its esters: an emerging treatment for multiple sclerosis, Curr Neuropharmacol, 7: 60-64, 2009; Linker et al., Fumaric acid esters exert neuroprotective effects in neuroinflammation via activation of the Nrf2 antioxidant pathway. Brain 134: 678-692, 2011). In fact, an interesting property that was also largely elucidated on preclinical grounds is the ability of DMF to positively impact the natural anti-oxidative stress machinery of cells. In resting states, nuclear factor (erythroid derived 2)-like2 (NRF2), the major transcription factor for genes involved in anti-oxidative responses, is sequestered in the cytoplasm by the Kelch-like erythroid cell-derived (ECH) associated protein-1 (KEAP-1). MMF has been shown to bind to KEAP-1 and enable the nuclear translocation of NRF2, resulting in transcription of anti-oxidative genes such as hemoxygenase-1 (HMOX1), nicotinamide adenine dinucleotide phosphate (NADPH), quinoline oxidoreductase-1 (NQO1) and others (Chen et al. Hydroxycarboxylic acid receptor 2 mediates dimethyl fumarate's protective effect in EAE. J Clin Invest 124: 2188-2192. 2014).

While the details of the interaction between DMF, its membrane receptor and the downstream events continue to be unveiled (Chen et al. Hydroxycarboxylic acid receptor 2 mediates dimethyl fumarate's protective effect in EAE. J Clin Invest 124: 2188-2192. 2014), a key message has already emerged: quite remarkably, the biological effects of DMF on the NRF2 pathway are what mediates its immune regulatory properties and lend to the implication that DMF has the potential for being a cytoprotecting agent, a role that at least in animals DMF has been proven to exert (Lastres-Becker et al., Repurposing the NRF2 Activator of Dimethyl Fumarate as Therapy Against Synucleinopathy in Parkinson's Disease, Antioxidants & Redox Signaling, 25(2), 2016, DOI: 10.1089/ars.205.6549; Scannevin et al., Fumarates Promote Cytoprotection of Central Nervous System Cells against Oxidative Stress via the Nuclear Factor (Erythroid-Derived 2)-Like 2 Pathway. J Pharmacology and Experimental Therapeutics April 2012, 341 (1) 274-284; DOI: ttps://doi.org/10.1124/jpet.111.190132; Fox et al., BG-12 (dimethyl fumarate): a review of mechanism of action, efficacy, and safety, Curr Med Res Opin 2014 Feb. 22; 30(2):251-62, 2014); Matolcsi & Rosza, Extending therapeutic possibilities in relapsing-remitting multiple sclerosis: dimethyl fumarate, Ideggyogy Sz, 68(1-2):7-14, 2015; Cross and Smith, Established and novel disease-modifying treatments in multiple sclerosis, J Internal Medicine, 275(4):350-363, 2014).

A comparison of orally administered disease modifying therapies for MS as established by Cross and Smith (Established and novel disease-modifying treatments in multiple sclerosis, J Internal Medicine, 275(4):350-363, 2014) and presented in Table 1 reveals that DMF has the desired attributes over the other two. Indeed, as shown, it is now known that Fingolimod may cause cardiac arrest (Vergas & Perumal, Fingolimod and cardiac risk: latest findings and clinical implications, Ther Adv Drug Saf, 4(3):119-124, 2013) and questions of potential cardiovascular death has been raised for Teriflunomid (Product Monograph—Genzyme Canada, Submission Control #178643; dated Sep. 18, 2015).

TABLE 1
Orally administered disease modifying therapies for MS

| | Relapse rate | | | Gadolinium | | |
|---|---|---|---|---|---|---|
| | reduction vs. | Disability | T2w lesion | enhancement | | |
| Therapy | placebo, % | reduction, % | reduction, % | reduction, % | Side effects | Monitoring |
| Fingolimod | 53 | 30 | 74 | 82 | First dose | Baseline anti-varicella |
| | | | | | bradycardia, | zoster IgG, pregnancy |
| | | | | | increased blood | test, blood pressure, |
| | | | | | pressure, | EKG. Baseline and |
| | | | | | macular edema, | Ongoing; blood counts, |
| | | | | | increased liver | liver enzymes, |
| | | | | | enzymes, | ophthalmology exam |
| | | | | | infections | baseline and 3 months |
| Teriflunomide | 31-36 | 30-31 | 67 | 80 | Gl, hair thinning, | Baseline blood counts, |
| | | | | | leukopenia. | liver enzymes, |
| | | | | | elevated liver | Tuberculosis test, |
| | | | | | enzymes, | pregnancy test, blood |
| | | | | | infections, | pressure. Monthly liver |
| | | | | | increased blood | enzymes for first |
| | | | | | pressure | 6 months. Biannual |
| | | | | | Pregnancy | blood counts and liver |
| | | | | | category X within | enzymes. |
| | | | | | US. | |
| Dimethyl fumarate | 44-53 | 38 | 71-85 | 74-90 | Flushing, | Baseline blood counts, |
| | | | | | diarrhea, | liver enzymes with |
| | | | | | abdominal pain, | periodic retesting as |
| | | | | | vomiting, | indicated |
| | | | | | lymphopenia, | |
| | | | | | elevated hepatic | |
| | | | | | enzymes. | |

Fumaric acid esters have an immunomodulatory mechanism of action with demonstrated efficacy for treatment of autoimmune diseases such as psoriasis and multiple sclerosis [Zecca C., Caporro M., Adami M., Mainetti C. and Gobbi C. Fumaric acid esters in psoriasis and multiple sclerosis. Clinical and experimental dermatology 39(4):488-491, 2014]. Dimethyl fumarate (DMF) is a fumaric acid ester (FAE) which has been approved as an oral therapy for relapsing forms of multiple sclerosis (MS) and has been shown to be effective in significantly reducing clinical and radiologic measures of disease activity in patients with relapsing remitting MS (RRMS) in clinical trials as well as in patients with MS treated in real-world studies. DMF undergoes esterase cleavage to monomethyl fumarate (MMF) prior to reaching systemic circulation in the blood. DMF therapy is thought to act by modulating cell-signaling pathways that produce neuroprotective and immunomodulatory effects. The effectiveness of DMF in the setting of multiple sclerosis, particularly RRMS, was initially demonstrated by two phase Ill trials (DEFINE and CONFIRM) [Fox R. J., Miller D. H., Phillips J. T., Hutchinson M., Havrdova E., Kita M., Yang M., Raghupathi K., Novas M., Sweetser M. T., Viglietta V. and Dawson K. T. Placebo-controlled phase 3 study of oral BG-12 or glatiramer in multiple sclerosis. The New England journal of medicine 367(12):1087-1097, 2012; Giralt M., Ramos R., Quintana A., Ferrer B., Erta M., Castro-Freire M., Comes G., Sanz E., Unzeta M., Pifarré P., Garcia A., Campbell I. L. and Hidalgo J. Induction of atypical EAE mediated by transgenic production of IL-6 in astrocytes in the absence of systemic IL-6. Glia 61(4):587-600, 2013]. These trials demonstrated reduced rates of disease relapse among adults (ages 18-55) with recent disease activity. In both trials, flushing was the most reported adverse effect (24-38%), although specific gastrointestinal (GI) adverse effects, including nausea, vomiting, diarrhea, and upper abdominal pain, occurred in 7 to 19% of patients. It has subsequently been reported that 36% of patients in these trials experienced adverse GI effects after 1 year, and in real-world settings GI side effects led to discontinuation of DMF in 5-19% of patients [Min J., Cohan S., Alvarez E., Sloane J., Phillips J. T., van der Walt A., Koulinska I., Fang F., Miller C. and Chan A. Real-World Characterization of Dimethyl Fumarate-Related Gastrointestinal Events in Multiple Sclerosis: Management Strategies to Improve Persistence on Treatment and Patient Outcomes. Neurology and therapy 8(1):109-119, 2019; Vollmer B., Nair K. V., Sillau S. H., Corboy J., Vollmer T. and Alvarez E. Comparison of fingolimod and dimethyl fumarate in the treatment of multiple sclerosis: Two-year experience. Multiple sclerosis journal—experimental, translational and clinical 3(3):2055217317725102, 2017]. Other studies report even higher incidence of GI issues with up to 40% reporting GI issues and up to 88% in real world self-reporting studies. [Palte et. al., Improving the Gastrointestinal Tolerability of Fumaric Acid Esters: Early Findings on Gastrointestinal Events with Diroximel Fumarate in Patients with Relapsing-Remitting Multiple Sclerosis from the Phase 3, Open-Label EVOLVE-MS-1 Study, Adv. Therapy, 36:3154-3165 (2019)] Ultimately, these adverse effects have led many MS suffers to stop DMF and seek alternate therapies. Still further, though rare, isolated instances of progressive multifocal leukoencephalopathy (PML) and lymphocytopenia, particularly in the first year of treatment, have been noted. [Linker and Haghikia, Dimethyl fumarate in multiple sclerosis: latest developments, evidence and place in therapy, Ther Adv Chronic Dis 7(4):198-207, 2016; Prosperini and Pontecorvo, Dimethyl fumarate in the management of multiple sclerosis: appropriate patient selection and special considerations, Ther Clin Risk Manag. 12:339-350, 2016].

Recent efforts to identify suitable alternatives to DMF have led to the discovery of diroximel fumarate (DRF). DRF undergoes esterase cleavage in the GI tract to generate monomethyl fumarate (MMF), 2-hydroxyethyl succinimide (HES), RDC-8439 (diroximel fumaric acid metabolite obtained due to demethylation of ester) and methanol, though at a considerably lower level than DMF [Jonasson E. and Sejbaek T. Diroximel fumarate in the treatment of multiple sclerosis. Neurodegenerative disease management 10(5):267-276, 2020]. MMF is believed to be the active metabolite of DMF and DRF. 462 mg and DMF 240 mg given twice-daily (BID) appear bioequivalent in terms of metabolic MMF generation. A 96-week study (EVOLVE-MS-1) showed that 30.9% of patients given DRF 462 mg BID still developed adverse GI effects, but such effects led to discontinuation in only 6.3% of patients [60]. Consistent with this, a 5-week study showed that the number of days with at least moderate GI symptoms is reduced by 46% in patients given DRF 462 mg BID as compared to DMF 240 mg BID [Naismith R. T., Wundes A., Ziemssen T., Jasinska E., Freedman M. S., Lembo A. J., Selmaj K., Bidollari I., Chen H., Hanna J., Leigh-Pemberton R., Lopez-Bresnahan M., Lyons J., Miller C., Rezendes D. and Wolinsky J. S. Diroximel Fumarate Demonstrates an Improved Gastrointestinal Tolerability Profile Compared with Dimethyl Fumarate in Patients with Relapsing-Remitting Multiple Sclerosis: Results from the Randomized, Double-Blind, Phase III EVOLVE-MS-2 Study. CNS drugs 34(2):185-196, 2020].

The improved tolerability of DRF as opposed to DMF appears to be related to its unique chemical structure, which is mostly (>90%) metabolized to HES and MMF, with RDC-8439 and methanol being generated as minor products (<10%) [Jonasson E. and Sejbaek T. Diroximel fumarate in the treatment of multiple sclerosis. Neurodegenerative disease management 10(5):267-276, 2020]. The generation of high methanol concentrations in the small intestine is likely responsible for significant level of adverse GI effects seen with DMF treatment. Though similarly generated with the use of DRF, the lower concentration of methanol found with DRF treatment is likely the basis for its reduced degree of gastrointestinal AE and greater tolerability in MS patients. [Palte M. J., Wehr A., Tawa M., Perkin K., Leigh-Pemberton R., Hanna J., Miller C. and Penner N. Improving the Gastrointestinal Tolerability of Fumaric Acid Esters: Early Findings on Gastrointestinal Events with Diroximel Fumarate in Patients with Relapsing-Remitting Multiple Sclerosis from the Phase 3, Open-Label EVOLVE-MS-1 Study. Advances in therapy 36(11):3154-3165, 2019]. [0014]Given the limited options, limited efficacy and the undesirable side-effects of those options, there has been and continues to be an important and urgent need for new therapeutic strategies, especially those that offer greater efficacy and patient satisfaction and a safer and more patient tolerable/acceptable risk profile in order to optimize therapeutic outcome. Effective, safe, and well-tolerated therapies can improve compliance and empower patients with a level of independence not presently possible.

In following, particularly given the apparent benefit of fumaric acid esters in the treatment of MS, there has been and continues to be a strong and urgent need to develop new and especially improved fumaric acid ester MS therapies as an alternative to existing medicaments, particularly the known therapies based on or prodrugs for fumaric acid esters.

In particular, there is a need for new and/or improved oral treatments, especially those that manifest a reduction/elimination in the occurrence of adverse events, especially lymphocytopenia, flushing and/or gastro-intestinal related side effects; have improved and/or longer/long-term efficacy; and are devoid of skin sensitization, thereby increasing ease of handling.

### SUMMARY OF THE INVENTION

In accordance with the present disclosure there are provided novel compositions and methods for ameliorating, reducing and/or reversing the effects and/or manifestation of multiple sclerosis as well as other disease conditions that share common mechanistic pathways. Specifically, it has now been found that compounds according to the general formula (I):

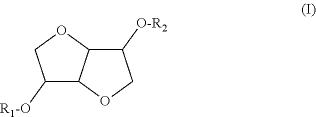

in which R₁ and R₂ are both —C(O)CH═CH—C(O)OMe or one of R₁ and R₂ is H and the other —C(O)CH═CH—C(O)OMe, wherein Me is methyl, especially the mono- and di-fumarate esters, formulae (I)(a) and (I)(b), respectively, whose structures are as follows:

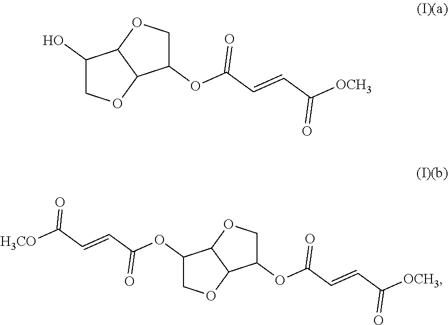 [SMILES: COC(=O)/C=C/C(=O)OC1COC2C(O)COC12 ; COC(=O)/C=C/C(=O)OC1COC2C(OC(=O)/C=C/C(=O)OC)COC12]

are effective in the treatment of multiple sclerosis and other disease conditions that share common gene expressions. Especially preferred are isosorbide di-(methyfumarate) (IDMF) and isosorbide mono-(methylfumarate) (IMMF). IDMF and IMMF have been found to be multi-targeted/targeting compounds which simultaneously affect various factors/conditions of the MS and related diseases. Though not intended to be bound by theory, it is believed that these compounds act, at least in part, through various potent anti-inflammatory and antioxidant pathways as well as pathways associated with regeneration and/or protection of myelination and/or interference with multiple sclerosis pathogenesis. Additionally, unlike DMF, IDMF is devoid or comparatively devoid of skin sensitization and unlike DMF and DRF, Applicant has seen no evidence of nor have they any reason to believe that methanol would be generated during metabolism of IDMF in the gastrointestinal tract: thereby avoiding the intestinal issues found with DMF and DRF administration.

Furthermore, the compounds of Formula I can be formulated with or into various known carriers and/or treatment compositions and can be administered by any of the known or yet to be discovered pharmaceutical methods of applications, e.g., oral, intramuscular, intravenous, transdermal, as well as sustained/timed release dosing. In following, while these compounds are effective individually, they can be used in combination with each other, e.g., combinations of two or more compounds meeting Formula I above, or in combination with other pharmacologically active compounds, particularly compounds that reduce, ameliorate, inhibit or otherwise address or treat MS symptoms and/or conditions associated with MS and its concurrent diseases/maladies.

The compositions of the present teaching can be administered through appropriate means, e.g., oral, subcutaneous, etc., with appropriate carriers or vehicles in the treatment of those suffering from MS and/or showing early signs of MS or possible MS development as well as a precautionary treatment to those who are predisposed to MS.

### DETAILED DESCRIPTION OF THE FIGURES

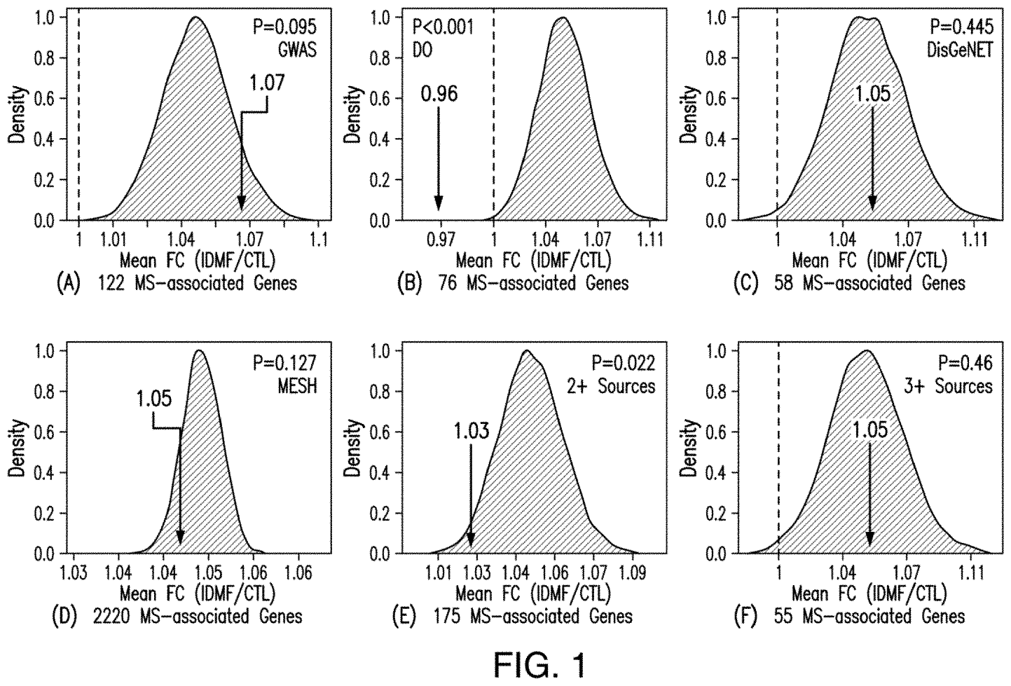

FIG. 1 presents histograms of MS-associated genes and their IDMF response.

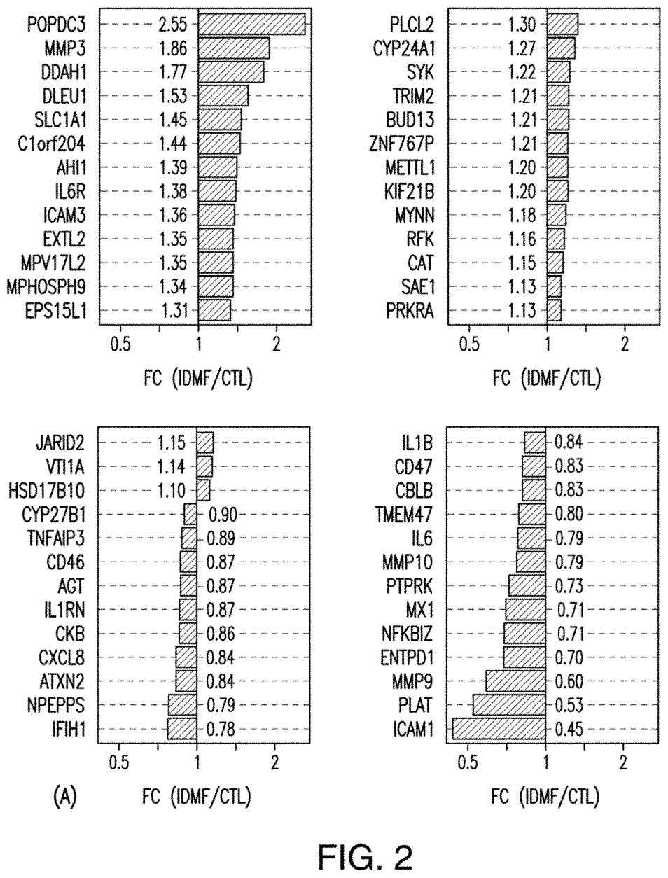

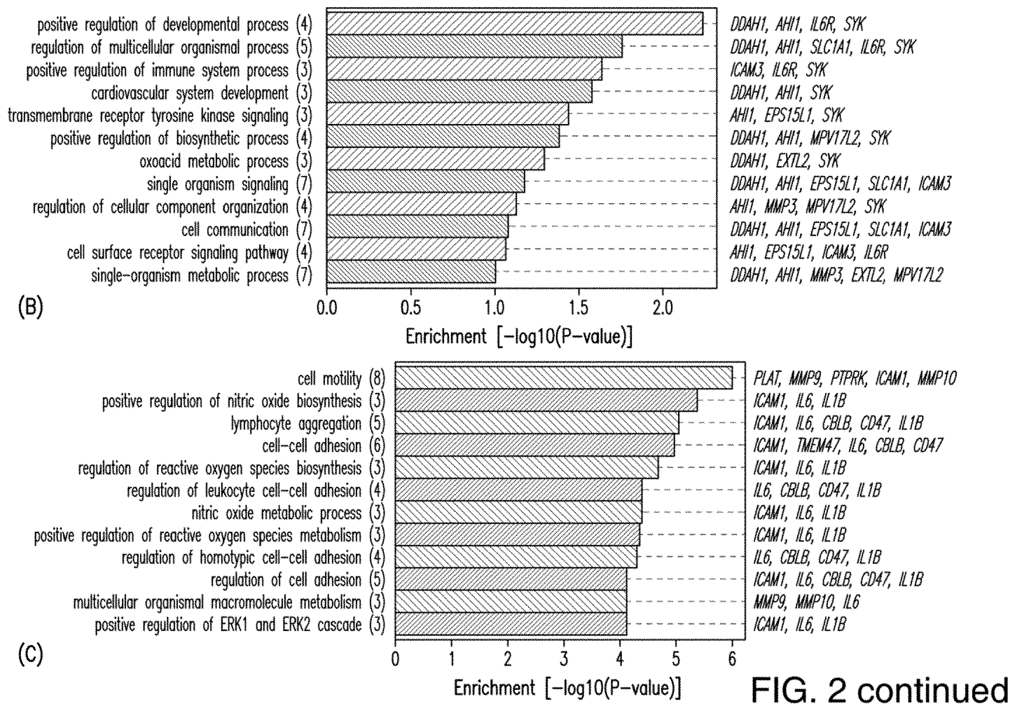

FIG. 2 presents histograms of IDMF influenced MS-associated genes.

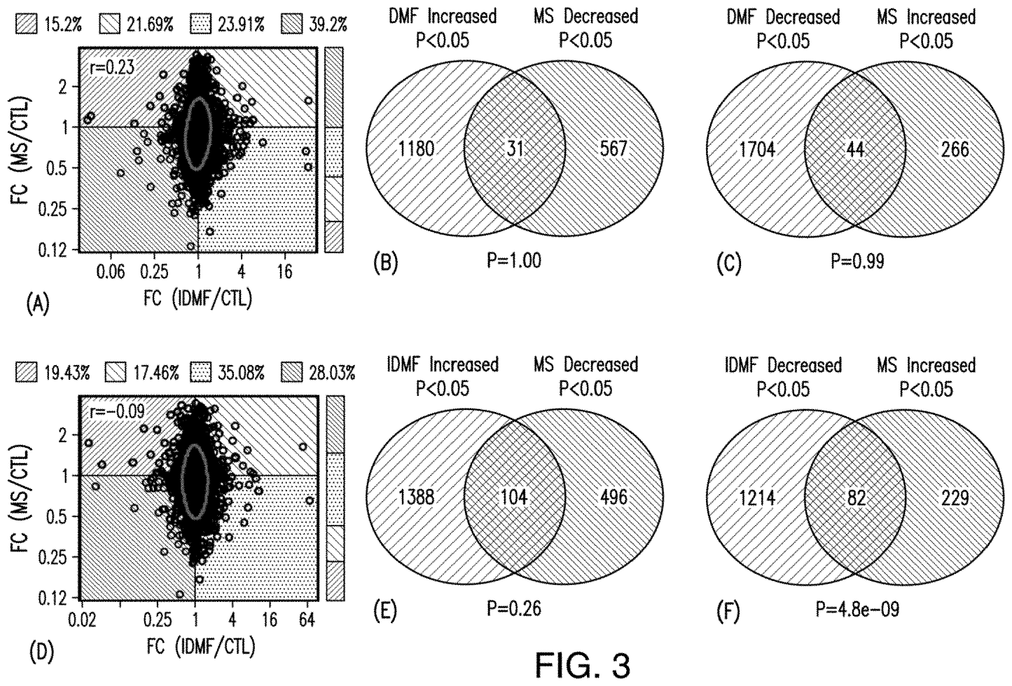

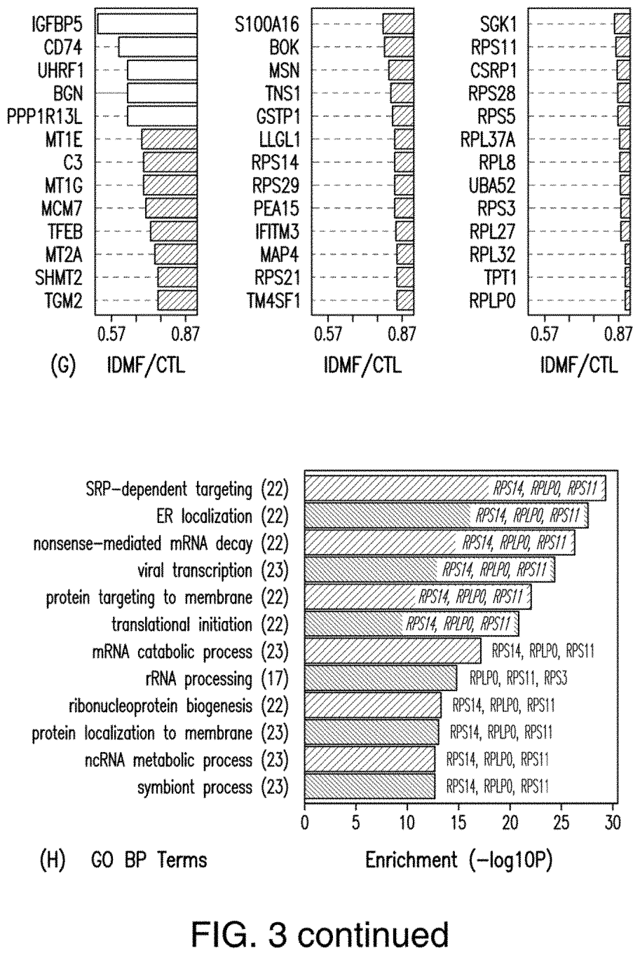

FIG. 3 presents scatterplots and histograms comparing the effects of IDMF and DMF on MS patient white matter astrocytes (GSE83670).

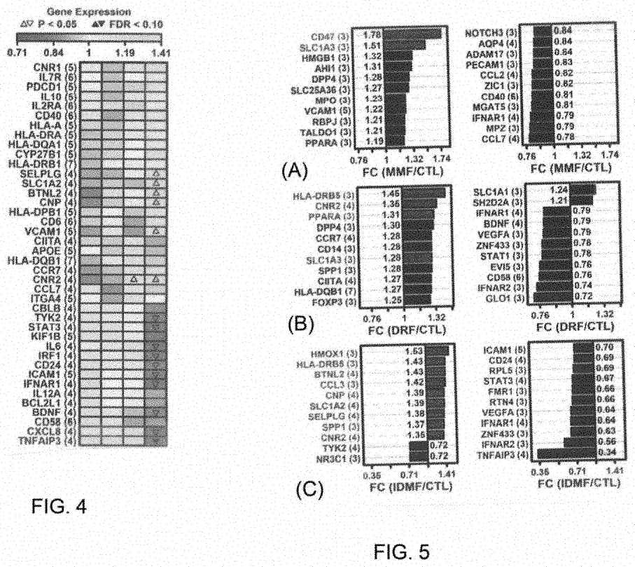

FIG. 4 presents a chart of various MS associated genes and their response to IDMF, DRF and MMF.

FIGS. 5(A), 5(B) and 5(C) present bar charts of the MS associated genes most significantly altered by MMF, DRF and IDMF respectively.

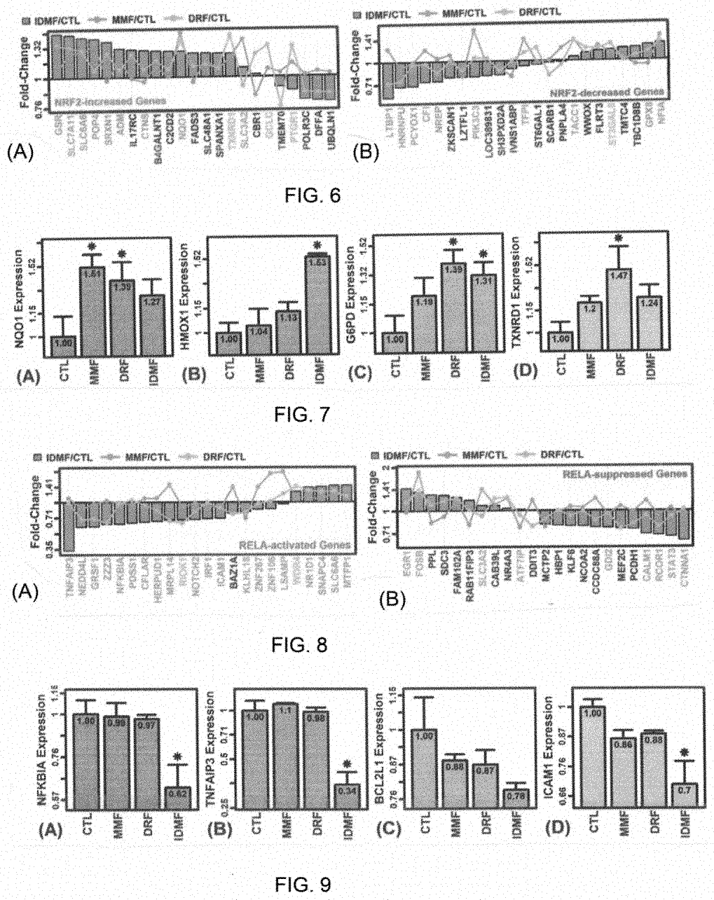

FIGS. 6(A) and 6(B) present hybrid charts and graphs of the impact of IDMF, MMF and DRF on NFR2-increased and NGR2-decreased target genes, respectively.

FIGS. 7(A) through 7(D) present bar charts of the impact of IDMF, MMF and DRF on specific NFR2 target genes.

FIGS. 8(A) and 8(B) present hybrid charts and graphs of the impact of IDMF, MMF and DRF on NF-kB-increased and NF-kB-decreased target (RELA) genes, respectively.

FIGS. 9(A) through 9(D) present bar charts of the impact of IDMF, MMF and DRF on specific NF-kB (RELA) target genes.

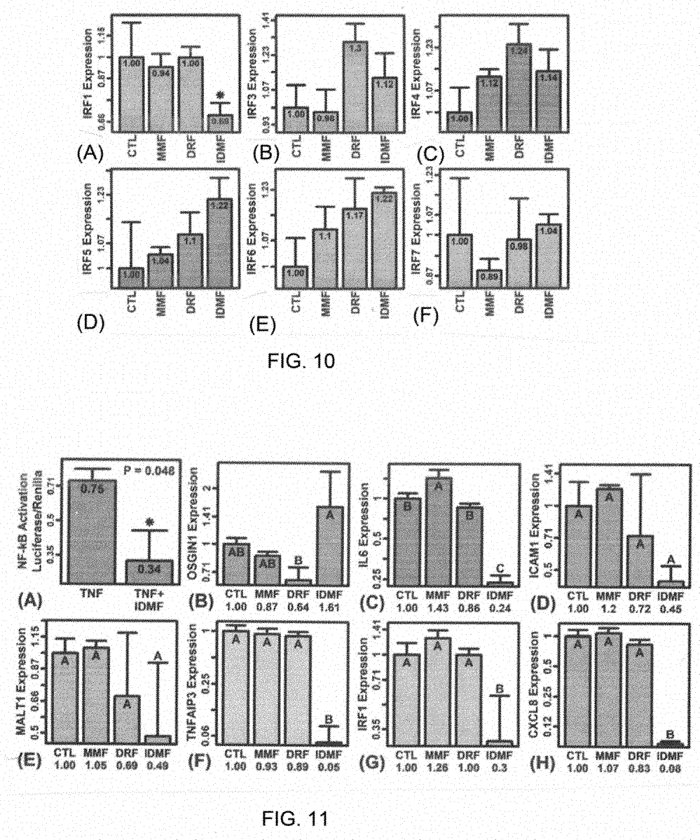

FIGS. 10(A) through 10(F) present bar charts of the impact of IDMF, MMF and DRF on the expression of different interferon regulatory factors.

FIGS. 11(A) through 11(H) presents bar charts of the impact of IDMF, MMF and DRF on specific key MS associated genes.

The file of this patent contains a least one drawing executed in color. Copies of this patent with color drawings will be provided by the United States Patent and Trademark Office upon request and payment of the necessary fee.

### DETAILED DESCRIPTION OF THE INVENTION

As used in the present specification, the following terms shall have the meanings as presented:

“Patient” refers to a mammal, for example, a human.

“Pharmaceutically acceptable” means that the subject of this descriptor has been approved or is otherwise approvable by a regulatory agency of a government or governmental or is listed in the U.S. Pharmacopoeia or other generally recognized pharmacopoeia for use in animals, and more particularly in humans.

“Pharmaceutically acceptable vehicle” refers to a pharmaceutically acceptable diluent, a pharmaceutically acceptable adjuvant, a pharmaceutically acceptable excipient, a pharmaceutically acceptable carrier, or a combination of any of the foregoing with which a pharmacological active agent, including the compounds provided by the present disclosure, can be administered to a patient, which does not destroy or have a marked adverse effect on the pharmacological activity of the therein contained pharmacological active agent or metabolite thereof and which is non-toxic when administered in doses sufficient to provide a therapeutically effective amount of the pharmacological active agent or metabolite thereof.

“Pharmaceutical composition” refers to a composition comprising a pharmaceutically acceptable vehicle and a pharmacological active agent or metabolite, especially, in the case of pharmaceutical compositions claimed by the present application, pharmacological actives of Formula (I).

“Preventing” or “prevention” of any disease refers to reducing the risk of acquiring the disease, as through the use of a pharmacological active agent as a vaccine.

“Treating” or “treatment” of any disease refers to reversing, alleviating, arresting, inhibiting, or ameliorating a disease or at least one of the clinical symptoms of a disease, inhibiting the progress of a disease or at least one of the clinical symptoms of the disease as well as delaying the onset of a disease or at least one or more symptoms thereof in a patient who is predisposed to a disease, especially as evidenced by genetic testing, even though that patient does not yet experience or display symptoms of the disease. In following, treating or treatment also refers to inhibiting a disease, either physically, (e.g., stabilization of a discernible symptom), physiologically, (e.g., stabilization of a physical parameter), or both, and to inhibiting at least one physical parameter that may or may not be discernible to the patient.

“Improve” or “improvement” is used to convey the fact that the pharmacological active agent has manifested or effected changes, most notably beneficial changes, in either the characteristics and/or the physical attributes of the tissue to which it is being provided, applied or administered. These terms are also used to indicate that the symptoms or physical characteristics associated with the diseased state are diminished, reduced or eliminated.

“inhibiting” generally refers to delaying the onset of the symptoms, delaying or stopping the progression of the symptoms, alleviating the symptoms, or eliminating the disease, condition or disorder.

“Optional” or “optionally” means that the subsequently described subject, event or circumstance is not required or a necessary consequence, and that the description includes instances where the event occurs and instances where it does not and/or when the subject is present and when it is not present.

“Therapeutically effective amount” refers to the amount of a compound or composition that, when administered to a patient for treating a disease, or at least one of the clinical symptoms of a disease, is sufficient to effect such treatment of the disease or symptom thereof. The “therapeutically effective amount” varies depending, for example, on the compound or composition, the disease and/or symptoms of the disease, the severity of the disease and/or symptoms of the disease, the age, weight, and/or health of the patient to be treated, and the judgment of the prescribing physician. An appropriate amount of any given compound or composition can be ascertained by those skilled in the art and/or is capable of determination by routine experimentation.

“Therapeutically effective dose” refers to a dose that provides effective treatment of a disease in a patient. A therapeutically effective dose varies from compound/composition to compound/composition and/or from patient to patient, and depends upon factors such as the condition of the patient and the route of delivery as well as those described in the preceding definition of therapeutically effective amount. A therapeutically effective dose can be determined in accordance with routine pharmacological procedures known to those skilled in the art.

Erring on the side of caution and in an effort to avoid having overlooked or inadvertently omitted certain descriptive matter, particularly complementary and supplementary descriptive matter, Applicant hereby states and affirms that the technical publications as well as the patent and patent application publications mentioned herein are all incorporated herein in their entirety by this reference. Indeed, for example, while Applicant could present page after page of description of suitable pharmaceutically acceptable vehicles, such would not be productive as the same are well known and well recognized by those skilled in the art and those that come into being subsequent to the filing of this application will readily be appreciated as suitable as well. The same holds true for many other potential constituents, both active and non-active, that are employed in pharmaceutical compositions made in accordance with the present teachings.

In accordance with a first aspect of the present disclosure there are provided novel compositions for the treatment of multiple sclerosis and related diseases, comprising one or more compounds according to the general Formula (I):

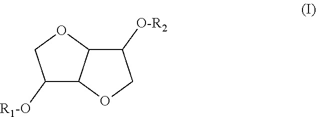

in which R₁ and R₂ are both —C(O)CH═CH—COOMe or one of R₁ and R₂ are H and the other —C(O)CH═CH—COOMe wherein Me is methyl, especially the mono- and di-fumarate esters, formulae (I)(a) and (I)(b), respectively, whose structure are as follows:

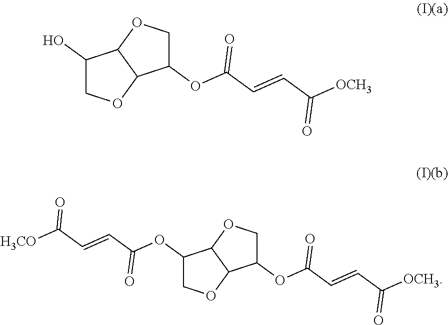 [SMILES: COC(=O)/C=C/C(=O)OC1COC2C(O)COC12 ; COC(=O)/C=C/C(=O)OC1COC2C(OC(=O)/C=C/C(=O)OC)COC12]

Especially preferred are isosorbide di-(methyfumarate) (IDMF) and isosorbide mono-(methylfumarate) (IMMF). It is to be appreciated that in the above Formula (I), the structural orientation of the —OR₁ and —OR₂ groups can be in an endo orientation (an isomannide), an exo orientation (an isoidide) or one can be endo and the other exo (an isosorbide). Owing to their structure, the isomannide and isoidide compounds are both symmetrical molecules; whereas, because isosorbide has one endo and one exo group, mono-acylation gives rise to two different non-equivalent ester products, namely a 2-ester or a 5-ester. Generally speaking, it has been found that these compounds have the characteristics of bis secondary alcohols attached to two cis-fused tetrahydrofuran rings and as such possess the properties of both diols and ethers. The preferred compound according to Formula (I) is the isosorbide di-(methyl fumarate) (IDMF) whose structure (IV) is given below:

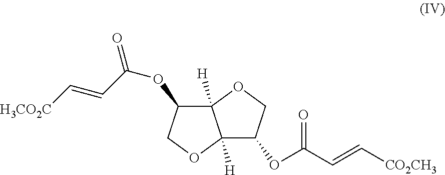 [SMILES: COC(=O)/C=C/C(=O)O[C@H]1CO[C@H]2[C@@H]1OC[C@H]2OC(=O)/C=C/C(=O)OC]

The compounds of Formula (I) are derived from dianhydrohexitols, which are well documented by-products of the starch industry obtained by dehydration of D-hexitols, which are made by a simple reduction of hexose sugars. About 650,000 tons of dianhydrohexitols are produced annually worldwide. These chiral biomass-derived products exist as three main isomers (isosorbide (V), isomannide (VI), and isoidide (VII)), depending on the configuration of the two hydroxyl functions (derived from D-glucose, D-mannose, and L-fructose, respectively). Isosorbide, which is produced from glucose via sorbitol, is the most widely available dianhydrohexitol.

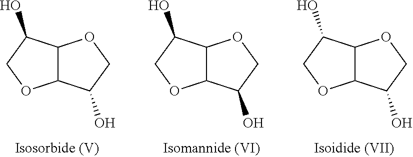

These dianhydrohexitols compounds, as well as the lower (C1-C4) mono- and di-alkyl ethers thereof, and the mono and di-nitrates thereof, are well known and already used in various medical, pharmaceutical and health and beauty applications. The unsubstituted and lower alkyl substituted isohexides are very soluble in water and biologically harmless. The lower alkyl ethers and the unsubstituted compounds have been used as carriers in a number of skin care products to aid in the transport of other active ingredients through the skin membrane. The lower alkyl ethers have also found utility in dentifrices, aiding in the removal of plaque due to their osmotic properties. Isosorbide dinitrate and isosorbide mononitrate have been used to treat angina pectoris. Like other nitric oxide donors, these drugs lower portal pressure by vasodilation and decreasing cardiac output.

Certain higher alkyl (C6 and higher) mono- and/or di-esters of the dianhydrohexitols have drawn significant scientific and commercial interest. For example, isosorbide di-caprylate (HydraSynol® DOI) and isosorbide di-(linoleate/oleate) (HydraSynol® IDL), both from Sytheon Ltd., have recently been shown to have skin hydration and barrier building properties and are used in skin care and treatment products (See e.g. Chaudhuri U.S. Pat. No. 8,496,917 and Chaudhuri, U.S. Ser. No. 15/277,990, filed Sep. 22, 2016, respectively). Similarly, compounds of Formula (I) have also been shown to be effective in the treatment of psoriasis (See Chaudhuri et. al. US Patent Publication 2016/0279092).

In accordance with the present teachings, compounds according to Formula (I) have, surprisingly, been found to be very effective in the treatment of multiple sclerosis and related diseases and may also be suitable for use in the prevention of MS. In this respect, IDMF and IMMF have been found to be multi-targeted/targeting compounds which simultaneously affect various factors/conditions of MS. Additionally, IDMF is devoid of skin sensitization and flushing: issues plaguing DMF as an effective MS treatment. Although these compounds can be used as is, they are preferably formulated with or into suitable pharmaceutically acceptable vehicles and/or pharmaceutical compositions: vehicles and/or compositions that are specially formulated for these compounds or vehicles and compositions that are well known and/or used for administering pharmacological active agents. Similarly, these compounds and the compositions containing the same can be administered by any suitable means or method depending upon the nature of the composition itself and the intended target in the patient. For example, the compounds of the present teaching can be administered in accordance with various methods of application, e.g., oral, intramuscular, intravenous, transdermal, formulations, as the sole active or as one of several actives, which act independently or in conjunction with one another as well as in formulations that allow for sustained/timed release dosing.

As noted above, the compounds of Formula (i), especially the dianhydrohexitol di-(methylfumarate), most especially isosorbide di-(methylfumarate) (IDMF), has surprisingly been found to provide a marked effect in ameliorating, reducing, delaying and/or reversing or otherwise treating the effects and/or manifestation of MS as well as other disease manifesting similar symptoms and/or sharing common gene expression profiles, at least with respect to those genes that appear to be disease related. While not intending to be bound by theory or mechanisms, it is believed that these compounds, especially IDMF, is capable of modulating key genes/proteins related to NRF2 pathway in MS involving numerous immune axes, particularly various arms of the T-lymphocyte axis, and elevated oxidative stress, including impaired antioxidant responses.

While the compounds of Formula (I) are effective individually, they can be, and are preferably and/or beneficially, used in combination with each other, e.g., combinations of two or more compounds of Formula (I), and/or in combination with other pharmacologically active compounds, particularly compounds that similarly reduce, ameliorate, inhibit or otherwise address or treat symptoms and/or conditions associated with MS as well as related diseases, specifically in association with other pharmacological active agents use to treat MS and/or other diseases often associated with or concurrently manifesting with MS. Such combinations of compounds and actives provide further surprising results in terms of their pharmacological activity, especially with respect to the treatment of MS and other diseases which manifest and/or have common effects on gene expression profiles. Such other pharmaceutical actives can be selected to treat the same disease or symptoms as the compounds of Formula (I) or a different disease or symptom. Alternate drugs useful for treating MS which can be combined with the compounds of Formula (I) or into which compounds of Formula (I) can be incorporated include, but are not limited to, Fingolimod, Teriflunomide, Dimethyl fumarate (DMF). Most especially, as noted above, the compounds of Formula (I) are incorporated into various pharmaceutical compositions for administration to a patient. These additional actives can be combined together and the combination of actives administered as a single pharmaceutical composition or administered independently, in concurrent or sequentially administered pharmaceutical compositions.

Thus, in accordance with yet another aspect of the teaching of the present disclosure there are provided pharmaceutical compositions and methods of treatment comprising a combination of two or more compounds according to Formula (I) as well as combinations of at least one compound of Formula (I) and one or more other suitable pharmaceutical active. Such combinations of active compounds and their application or administration is found to have improved and/or synergistic performance, particularly with respect to the treatment of MS and diseases which manifest similar symptoms and/or common gene expression profiles.

The pharmaceutical compositions provided by the present disclosure can be formulated in a unit dosage form. A unit dosage form refers to a physically discrete unit suitable as a unitary dose for patients undergoing treatment, with each unit containing a predetermined quantity of a compound of Formula (I) calculated to produce an intended therapeutic effect. A unit dosage form can be for a single daily dose, for administration 2 times per day, or one of multiple daily doses, e.g., 3 or more times per day. When multiple daily doses are used, a unit dosage form can be the same or different for each dose. One or more dosage forms typically comprise a dose, which can be administered to a patient at a single point in time or during a time interval.

The pharmaceutical compositions comprising a compound of Formula (I) can be formulated for immediate release or for delayed or controlled release. In this latter regard, certain embodiments, e.g., an orally administered product, can be adapted for controlled release. Controlled delivery technologies can improve the absorption of a drug in a particular region, or regions, of the gastrointestinal tract. Controlled drug delivery systems are designed to deliver a drug in such a way that the drug level is maintained within a therapeutically effective window and effective and safe blood levels are maintained for a period as long as the system continues to deliver the drug with a particular release profile in the gastrointestinal tract. Controlled drug delivery typically and preferably produces substantially constant blood levels of a drug over a period of time as compared to fluctuations observed with immediate release dosage forms. For some drugs, maintaining a constant blood and tissue concentration of the drug throughout the course of therapy is the most desirable mode of treatment as immediate release of drugs oftentimes causes blood levels to peak above that level required to elicit a desired response. This results in waste of the drug and/or may cause or exacerbate toxic side effects. In contrast, the controlled delivery of a drug can result in optimum therapy; not only reducing the frequency of dosing, but also reducing the severity of side effects. Examples of controlled release dosage forms include dissolution controlled systems, diffusion controlled systems, ion exchange resins, osmotically controlled systems, erodable matrix systems, pH independent formulations, and gastric retention systems.

As noted, the compounds of Formula (I), more appropriately, the pharmaceutical compositions comprising compounds of Formula (I), can be administered through any conventional method. The specific mode of application or administration is, in part, dependent upon the form of the pharmaceutical composition, the primary purpose or target of its application (e.g., the application may be oral if intending to address the disease generally or topically or injection if intending to address primarily a topical symptom or location of a symptom of the disease. Suitable modes of administration include, for example, intradermal, intramuscular, intraperitoneal, intravenous, subcutaneous, intranasal, epidural, oral, sublingual, intracerebral, intravaginal, transdermal, rectal, or inhalation. Especially preferred modes of administration are oral, or those methods that involve absorption through epithelial or mucous linings (e.g., oral mucosa, rectal, and intestinal mucosa, etc.). Furthermore, again, depending in part upon the form and primary purpose or target of the administration, the pharmaceutical compositions of the present disclosure can be administered systemically and/or locally. For example, hands, feet and legs are often a primary target of MS manifestation; hence, one may localize treatment to the hands, feet and/or legs to optimize dosing to those areas. Additionally, one may employ both systemic and local treatment to address MS systemically while concurrently localizing a stronger effect locally. Finally, the form of the pharmaceutical composition and its delivery system varies depending upon the parameters already noted. For example, orally administered pharmaceutical compositions of the present teaching can be in encapsulated form, e.g., encapsulated in liposomes, or as microparticles, microcapsules, capsules, etc.

Again as noted above, the compounds of Formula (I) can be used as is, i.e., as 100% of the composition to be applied; however, the compounds of Formula (I) are preferably incorporated into a pharmaceutical composition in which the compound(s) of formula account for from about 0.01 to about 99 weight percent of the pharmaceutical composition. Preferably, the compounds of Formula (I) will comprise from about 0.5 to about 30 wt %, more preferably from about 0.5 to about 20 wt %, most preferably from about 1.0 to about 10 wt % of the pharmaceutical composition. Another factor playing into the concentration of the compounds of Formula (I) in the pharmaceutical composition is the dose or rate of application of the compounds to the patient. Obviously, dosing itself depends upon a number of factors including the concentration and/or purity of the compounds of Formula (I), the efficacy thereof, the individual to whom the pharmaceutical is to be administered, the mode of administration, the form in which the pharmaceutical composition is to be administered, the disease or symptom to be addressed, etc.

The foregoing factors as well as the application thereof in formulating the compositions of the present teaching are all as well known in the art whereby the final or actual concentration in the pharmaceutical composition and/or the dose can readily be determined based up simple dose-response testing and the like. For example, an appropriate oral dosage for a particular pharmaceutical composition containing one or more compounds of formula (I) will depend, at least in part, on the gastrointestinal absorption properties of the compound, the stability of the compound in the gastrointestinal tract, the pharmacokinetics of the compound and the intended therapeutic profile.

An appropriate controlled release oral dosage and ultimate form of a pharmaceutical composition containing a particular compound of Formula (I) will also depend upon a number of factors. For example, gastric retention oral dosage forms may be appropriate for compounds absorbed primarily from the upper gastrointestinal tract, and sustained release oral dosage forms may be appropriate for compounds absorbed primarily from the lower gastrointestinal tract. Again, it is to be expected that certain compounds are absorbed primarily from the small intestine whereas others are absorbed primarily through the large intestine. It is also to be appreciated that while it is generally accepted that compounds traverse the length of the small intestine in about 3 to 5 hours, there are compounds that are not easily absorbed by the small intestine or that do not dissolve readily. Thus, in these instances, the window for active agent absorption in the small intestine may be too short to provide a desired therapeutic effect in which case large intestinal absorption must be channeled and/or alternate routes of administration pursued.

Generally speaking, an appropriate dose of a compound of Formula (I), or pharmaceutical composition comprising a compound of Formula (I), can be determined according to any one of several well-established protocols including in-vitro and/or in-vivo assays and/or model studies as well as clinical trials. For example, animal studies involving mice, rats, dogs, and/or monkeys can be used to determine an appropriate dose of a pharmaceutical compound. Results from animal studies are typically extrapolated to determine appropriate doses for use in other species, such as for example, humans.

As noted above, the compositions according to the present teaching can be designed for immediate infusion or application of the actives to the body or site of the symptom to be treated. However, it is also recognized that in certain instances the pharmaceutical compositions provided by the present disclosure can be, and are preferably, adapted to provide sustained or timed release of a compound of Formula (I): this is especially so and desirable for oral administration. Sustained release oral dosage forms are used to release drugs over a prolonged time period and are useful when it is desired that a drug or drug form be delivered to the lower gastrointestinal tract. Sustained release oral dosage forms include any oral dosage form that maintains therapeutic concentrations of a drug in a biological fluid such as the plasma, blood, cerebrospinal fluid, or in a tissue or organ for a prolonged time period. Sustained release oral dosage forms include diffusion-controlled systems such as reservoir devices and matrix devices, dissolution-controlled systems, osmotic systems, and erosion-controlled systems. Sustained release oral dosage forms and methods of preparing the same are well known in the art.

Following on the foregoing, the amount of a compound of Formula (I) contained in a dose depends upon, among other factors, the route of administration and whether the disease in a patient is effectively treated by acute, chronic, or a combination of acute and chronic administration. In any event, the administered dose is typically less than a toxic dose: though it may have significant adverse health effects, provided that the desired beneficial effect is also attained. Toxicity of the compositions described herein can be determined by standard pharmaceutical procedures in cell cultures or experimental animals, e.g., by determining the LD₅₀ (the dose lethal to 50% of the population) or the LD₁₀₀ (the dose lethal to 100% of the population). The dose ratio between toxic and therapeutic effect is the therapeutic index. In certain embodiments, a compound or metabolite thereof may exhibit a high therapeutic index. The data obtained from these cell culture assays and animal studies can be used in formulating a dosage range that is not toxic for use in humans. A dose of a compound of Formula (I) is typically set within a range of circulating concentrations in for example the blood, plasma, or central nervous system, that include the effective dose and that exhibits little or no toxicity. A dose can vary within this range depending upon the dosage form employed and the route of administration utilized. In certain embodiments, an escalating dose can be administered.

Where additional pharmacological actives may be and preferably are also present in the compositions according to the present teaching, the amount by which they are present and/or the dosage amount will typically be consistent with their conventional concentration and rates of application. For example, such other actives will be present in an amount of from about 0.5 to about 30 wt %, more preferably from about 0.5 to about 20 wt %, most preferably from about 1.0 to about 10 wt % of the pharmaceutical composition. Of course, as noted, the combination of these other pharmacological actives with one or more compounds of Formula (I) also provide enhanced performance and/or synergy whereby the amounts of each and/or the dose of each is generally less than required for the use of the individual active compounds on their own.

Without further elaboration, it is believed that one skilled in the art can, using the preceding description, utilize the present invention to its fullest extent. The following preferred specific embodiments are, therefore, to be construed as merely illustrative, and not limitative of the remainder of the disclosure in any way whatsoever.

### EXAMPLES

Experimental I

An initial series of experiments was performed to demonstrate the effect and comparative effect of IDMF and DMF in various MS related pathways.

### Example 1

A study was conducted using microarrays to evaluate the effects of IDMF on genome-wide expression in cultured human keratinocytes (KCs) in an effort to demonstrate the effect of IDMF on gene expression of genes associated with multiple sclerosis (MS) and, in particular, to demonstrate that IDMF induces the transcription of genes associated with nuclear factor (erythroid-derived 2)-like 2 (NFE2L2/NRF2), factors known to be associated with MS. RNA sample processing and microarray hybridizations were performed using the Affymetrix Human Genome U133 Plus 2.0 array platform. DNA microarray samples evaluated included IDMF- and vehicle-treated KCs (n=2 replicates per group). Samples were normalized using the robust multichip average (RMA) algorithm. Identification of probe sets with detectable expression was performed using the MAS 5.0 method, which identifies probe sets with above-background expression based upon the Wilcoxon signed rank test. To limit redundancy in downstream analyses, a single probe set was chosen to represent each protein-coding human gene featured on the microarray platform. This representative was chosen as the probe set with highest expression for each protein-coding human gene. These probe sets were then additionally filtered to include only those with detectable expression in at least 1 of the 4 samples (P<0.05, Wilcoxon signed rank test). Overall, this yielded a total of 12330 protein-coding genes that were included in analyses, with each gene represented by a single representative probe set. Bayesian linear models and moderated t-statistics were then used to evaluate differential expression for each of the 12330 genes (IDMF vs. CTL treatment; R package: limma). To control the false discovery rate (FDR), raw p-values generated from linear model analyses were adjusted using the Benjamini-Hochberg method.

MS Gene Association

Genes associated with multiple sclerosis were identified based upon four established database sources: (1) the NHGRI-EBI genome-wide association (GWA) study catalogue, (2) the Disease Ontology (DO) database, (3) the DisGeNET database, and (4) the Medical Subject Heading (MESH) database. While there is some overlap between the various databases, the variation in identified genes from one database to the other reflects the fact that the science is still developing and has uncertainties. Nevertheless, as discussed below, efforts were undertaken to hone in on genes common to multiple databases.

MS-associated genes identified from each of the four sources were evaluated to assess whether they exhibited atypical responses to IDMF, either exhibiting a trend towards increased or decreased expression as shown in the histograms of FIG. 1. For histograoms (A)-(F), the arrow indicates the average fold-change (IDMF/CTL) among MS-associated genes and the histogram represents the distribution of average fold-change estimates in randomly sampled gene sets (10,000 random samples for each analysis). As evident from the figures, the results varied depending upon the database and approach used to define the MS-associated genes. For the 76 MS-associated genes identified from the Disease Ontology (DO) database, the average fold-change (IDMF/CTL) was significantly lower than obtained in randomly sampled sets of 76 genes (FIG. 1, histogram B; P<0.001). Similarly, overlapping average fold change rates were noted with MESH identified MS-associated genes (FIG. 1, histogram D; P=0.127) and those MS-associated genes common to at least two of the four databases (FIG. 1, histogram E; P=0.022). On the other hand, for the 122 MS-associated genes identified from NHGRI-EBI genome-wide association (GWA) study catalogue a moderate to significant increase in average fold-change was noted as compared to the randomly sampled sets (FIG. 1, histogram A; P=0.095) and an equivalent average fold-change was noted in the case of the MS-associated genes of the DisGeNET database (FIG. 1,histogram C; P=0.445) and those MS-associated genes common to at least three of the four databases (FIG. 1, histogram F; P=0.46).

Of the 175 MS-associated genes identified as common to at least two of the four databases (FIG. 1, histogram E), the 52 genes that appeared to be the most influenced or impacted by IDMF (based on p-values) were selected for further evaluation. The impact of IDMF with respect to these genes is presented in the histograms of FIG. 2. Focusing on those genes on which IDMF had the most significant impact (FDR<0.10 andP>1), FIG. 2, histograms B and C present the Gene Ontology biological process terms enriched with respect to the 15 genes whose regulation was activity was increased the most by IDMF (namely POPDC3, MMP3, DDAH1, DLEU1, SLC1A1, C1orf204, AHI1, IL6R, ICAM3, EXTL2, MPV17L2, MPHOSPH9, EPS15L1, SYK and BUD13) and the 13 genes whose activity was decreased the most by IDMF (namely, IL1B, CD47, CBLB, TMEM47, IL6, MMP10, PTPRK, MX1, NFKBIZ, ENTPD1, MMP9, PLAT and ICAM1).

As indicated in the histograms of FIG. 2, MS-associated genes most strongly increased by IDMF included POPDC3, MMP3, and DDAH1, while MS-associated genes most strongly decreased by IDMF included ICAM1, PLAT, and MMP9. As evident from histogram B in FIG. 2, among the 15 IDMF-increased genes associated with MS there was significant enrichment for genes associated with development and immune system processes. As evident from histogram C in FIG. 2, among the 13 IDMF-decreased genes associated with MS by at least 2 database there was significant enrichment for genes associated with cell motility, nitric oxide and ROS synthesis, lymphocyte aggregation, and cell-cell adhesion. Such results correlate and are indicative of the effectiveness of IDMF in treating MS and its symptoms.

Nuclear Factor (Erythroid-Derived 2)-Like 2 (NFE2L2/NRF2) Study

As noted in the background section above, the transcription factor nuclear factor (erythroid-derived 2)-like 2 (NFE2L2/NRF2) has previously been associated with multiple sclerosis. In this study, IDMF-regulated genes were evaluated to determine the extent to which IDMF led to the enrichment of NFE2L2/NRF2 binding sites in promoter-proximal regions. For these analyses, a NFE2L2/NRF2 binding site was obtained from the JASPAR database (JASPAR ID: MA0150). This binding site partially matches the consensus sequence 5-[A/G]TGAC—AGCA-3: additional information can be obtained from http://jaspar.genereg.net/cgi-bin/jaspar_db.pl?ID=MA0150.1&rm=present&collection=CORE. Tests for enrichment were performed by scanning regions 1 kb, 2 kb or 5 kb upstream of protein-coding genes, including either all sequence (conserved=No) or only sequences conserved among mammalian species (conserved=Yes). A positive Z statistic indicates a trend towards increased NFE2L2 (NRF2) binding sites in sequences upstream of IDMF-increased Database Essential Genes (DEGs), as compared to all other expressed genes). P-values indicate the significance of the reported Z statistic (semiparametric generalized additive logistic models). Predicted targets include IDMF-increased DEGs with the NFE2L2 (NRF2) binding site identified within the scanned upstream region.

Results, as shown in Table 2, demonstrate that this NFE2L2/NRF2 binding site is significantly enriched in regions upstream of IDMF-increased DEGs (FDR<0.10 with FC>1.50). The enrichment tended to increase when sequences closer to the transcription start site were evaluated, and when only sequence regions conserved among mammalian species were analyzed. These trends suggest that the NFE2L2/NRF2 binding sites act as functional mediators of transcription for IDMF-increased genes.

The IDMF-increased genes with the NFE2L2/NRF2 binding site in close proximity (<1 kb) to their transcription start site include GSR, AIFM2, PIR, NQO1, CYP4F3, BEX5, GCLM, GPX2, and CYP4F11 (Table 2). The analysis was repeated with respect to IDMF-decreased genes (FDR<0.10 with FC<0.67), but in contrast such genes did not appear to show increased frequency of the NFE2L2/NRF2 binding site in their upstream region. Nevertheless, it should be noted that other binding sites were also identified showing a stronger degree of enrichment. For IDMF-increased genes (FDR<0.10 with FC>1.50), the strongest enrichment was observed for binding sites recognized by IRF1 (TWTTCA), LSM6 (ATGNARA) and HOXB5 (TAATTR). For IDMF-decreased genes (FDR<0.10 with FC<0.67), the strongest enrichment was observed for binding sites recognized by NF-kappaB (GGRAWTYCCC), MCTP2 (TTCCY) and FOXP4 (MTTTYCC). Although not a focal point of this analysis, it was noted that IDMF-decreased genes were especially and strongly enriched for NF-kappaB biding sites in their upstream regions (P=1.16E-08). IDMF-decreased genes associated with this binding site include IL23A, IL36G, ICAM1, and S100A7 and as associated with anti-inflammatory mechanisms which are believed to be especially beneficial in multiple disease states characterized by autoimmunity and/or inflammation: issues associated with MS. Indeed, as noted previously, it has been proposed that NF-kappaB provides a target for treatment of multiple sclerosis (PMID: 19128210; PMID: 24007818).

TABLE 2
Enrichment of NFE2L2 (NRF2) binding sites

| Region | Conserved | Z Statistic | P-value | Predicted Targets |
|---|---|---|---|---|
| 1 kb | No | 4.74 | 2.11E−06 | GSR, AIFM2, PIR, NQO1, CYP4F3, |
| | | | | BEX5, GCLM, GPX2, CYP4F11 |
| 2 kb | No | 3.37 | 0.00074 | AIFM2, NQO1, NAP1L2, GCLM, PIR, |
| | | | | BEX5, CLMP, CYP4F3, GPX2, CYP4F11 |
| 5 kb | No | 2.11 | 0.035 | NAP1L2, NQO1, CYP4F3, AIFM2, BEX5, |
| | | | | PIR, CLMP, GCLC, CYP4F11, CNNM4, |
| | | | | GPX2, SPP1, COL11A1 |
| 1 kb | Yes | 5.91 | 3.39E−09 | GSR, AIFM2, GPX2, BEX5, GCLM, PIR, |
| | | | | NQO1, CYP4F3, CYP4F11 |
| 2 kb | Yes | 3.21 | 0.0013 | NQO1, NAP1L2, GCLM, GPX2, PIR, |
| | | | | CYP4F3, CYP4F11 |
| 5 kb | Yes | 1.05 | 0.293 | NAP1L2, NQO1, GCLC, GPX2, |
| | | | | COL11A1, PIR |

### Example 2—IDMF/DMF

A separate assay study was conducted on cytokine-treated neonatal human epidermal keratinocytes (HEK) looking at the expression impact of IDMF and dimethyfumarate (DMF) on key pro-inflammatory and antioxidant genes. In preparation for the assay, HEK (p2; Cell Applications, San Diego) were cultured at optimal conditions [keratinocyte growth medium supplemented with KGM-Gold Bullet kit (Lonza, Switzerland)] to form a segmented monolayer. A cytokine mix of IL-17A/IL-22/TNFα (100 ng/ml; 100 ng/ml; 10 ng/ml; Antigenix, Huntington Station, N.Y.) was added and the cell cultures incubated for 24 h. Meanwhile the test formulations of IDMF and DMF were prepared by solubilizing each in DMSO to a concentration of 20 mg/ml and then diluting those solutions with water to a concentration of to 80 μg/ml. Thereafter, each test material was added to the cultures at 4 μg/ml (final concentration) in triplicate and the cultures incubated for a further 24 hours.

After the 24-hour incubation with the test materials, the cells were observed through a Nikon Eclipse TS100 inverted microscope. No gross morphological changes were recorded. RNA was then extracted and purified with NucleoSpin RNA II kit from Machery-Nagel, Bethlehem, Pa., using QiaCube robotic station (Qiagen, Valencia, Calif.). Purified total RNA was assessed at 260 nm and 280 nm with NanoDrop Lite (Thermo Fisher Scientific, Waltham, Mass.), pure samples with A₂₆₀A_{/280} and A₂₆₀/A₂₃₀ ratios of >1.7 were standardized and the expression of the genes of interest (Table 3) was measured by real-time quantitative PCR with BioRad iCycler iQ Detection System using custom-made PCR arrays (Qiagen, Valencia, Calif.; part #24caph12190), 5×All-In-One 1st Strand cDNA Synthesis Mix (Bioland Scientific, Paramount, Calif.) and 2×qPCR Master Mix (Bioland). Efficiency ΔΔCt method was used for quantification of results, after the normalization of gene expression to GAPDH/PPIA housekeeping gene. Genes were considered differentially expressed if the level of expression was reasonably high (less than 30 cycles to detect) and the modulation was >1.7. However, in noting the gene expressions attained, it is to be appreciated that given the difference in their molecular weights, when used at the same concentration (by wt) there is nearly 2.5 times as many moles of DMF as compared to IDMF. Even assuming IDMF fully metabolizes to MMF, there is still considerably more MMF from DMF as opposed to the same amount, based on weight, of IDMF. Hence, it is believed that had equivalent molar amounts of DMF and IDMF, based on available MMF, been used, the beneficial impact of IDMF would have been more apparent.

The results attained were as presented in Table 3. CXCL3, IL8 and PTGS2 all are genes associated with inflammation or pro-inflammatory effects. As noted in Table 3, DMF induced an upregulation in all three genes, indicative of an inflammatory inducing effect, whereas in the case of IDMF it is non-existent, if not of an anti-inflammatory effect, particularly with respect to CXCL3. Most significantly, IDMF had a markedly higher upregulation of NFE2L2 than DMF, even at the lower molar concentration. This is especially important with respect to the current claimed method since, as noted in the previous study, nuclear factor (erythroid-derived 2)-like 2, also known as NFE2L2 or Nrf2, is directly tied to MS treatment (Anna Hammer et al., The NRF2 pathway as potential biomarker for dimethyl fumarate treatment in multiple sclerosis, Ann Clin Transl Neurol, 5(6):668-676, 2018). Though not intended to be bound by theory, the mechanism of efficacy is believed to involve NFE2L2 (NRF2)-activated genes associated with glutathione metabolism and/or oxidative stress response. In this regard, NFE2L2 is a transcription factor that in humans is encoded by the NFE2L2 gene. It is a basic leucine zipper (bZIP) protein that regulates the expression of antioxidant proteins that protect against damage triggered by oxidative stress.

TABLE 3
Gene Expression IDMF v DMF

| | DMF 4 μg/ml | Detection | IDMF 4 μg/ml | Detection | |
|---|---|---|---|---|---|
| Gene name | fold change | level | fold change | level | Comments |
| CXCL3 | 1.7 | OKAY | −1.1 | OKAY | CXCL3 Chemokine (C-X-C motif) ligand 3 |
| | | | | | induces inflammation and is stimulated in |
| | | | | | keratinocytes by IL-17. |
| IL8 | 2.3 | OKAY | 1.4 | OKAY | Interleukin-8 (IL-8) is a pro-inflammatory |
| | | | | | cytokine in the skin. |
| PTGS2 | 1.7 | OKAY | 1.2 | OKAY | Prostaglandin-endoperoxide synthase |
| | | | | | (PTGS2), also known as cyclooxygenase 2 |
| | | | | | (COX2), is the key inducible enzyme in |
| | | | | | prostaglandin biosynthesis, and is involved |
| | | | | | in inflammation, contact dermatitis and |
| | | | | | mitogenesis. |
| NFE2L2 | 1.8 | OKAY | 2.6 | OKAY | NFE2L2 gene codes for the nuclear factor |
| | | | | | (erythroid-derived 2)-like (NRF2) - the |
| | | | | | transcription factor that enables the |
| | | | | | expression of many antioxidant proteins. |

### Example 3

A further study was conducted to evaluate the effects of DMF and IDMF on gene expression in cultured astrocytes. Previously, Waller et al. 2016 (J Neuroimmunol 299:139-146) used laser capture microdissection (LCM) to isolate astrocytes from normal appearing white matter in MS patients and CTL subjects (GEO dataset GSE83670; PMID: 27125112). The study was performed using 3 treatments and 6 independent biological replicates (CTL=non-stimulated astrocytes, n=2; DMF=DMF-treated astrocytes, n=2; IDMF=IDMF-treated astrocytes, n=2). Comparison of DMF/IDMF expression responses to MS vs. CTL expression changes from the Waller at al. 2016 study allows us to evaluate whether DMF/IDMF expression responses oppose those occurring in MS patients for the same cell type.

cDNA sequencing was performed by the University of Michigan sequencing core facility (50 cycle single end Illumina HiSeq 2000). The FASTX-Toolkit was used to filter reads by removing low quality sequences and initial quality assessments were performed using FastQC. Quality-filtered reads were mapped to the human genome (hg38/GRCh38, UCSC) using tophat2 with default settings, except multi-mapping of reads to multiple genome locations was disallowed (settings: -g 1 —no-coverage-search). The hg38/GRCh38 genome is the most recent release available from UCSC. HTSeq was used to tabulate the number of reads mapping to each genome feature and Cufflinks was used to calculate FPKM estimates and associated confidence intervals. RSeQC was used to assess quality of mapping and calculate the percentage of mapped reads. The negative binomial model (edgeR) was used to evaluate differential expression, with read counts normalized using the weighted trimmed mean of M-values method, and dispersions estimated using the Cox-Reid (CR)-adjusted likelihood approach. Differential expression analyses were performed using genes with detectable expression in at least 1 of 4 samples involved in a given comparison. A gene was considered to have detectable expression in a sample if the count per million mapped reads (cpm) estimate was greater than 0.20 and if the lower bound of the FPKM 95% confidence interval was greater than zero.

FIG. 3 presents in scatterplot and histogram format the results of the study on the astrocytes. Scatterplots (A) and (D) compare fold change (FC) estimates in DMF-treated (A) and IDMF-treated (D) astrocytes to LCM-dissected astrocytes in MS patients and healthy controls (CTLs). Each point represents a protein-coding gene and the light colored ellipses outline the 90% of genes closest to the bivariate mean based upon the Mahalanobis distance. The Spearman rank correlation coefficient estimate is shown (upper-left). The proportion of genes within each quadrant is indicated in the upper margin (P<0.05, Fisher's exact test), wherein the quadrants are defined by a vertical and horizontal line through the “1” in each axis with the proportion corresponding, left to right to the upper left, the upper right, the lower right and lower left quadrants, respectively. Gene set overlap is presented in (B), (C), (E) and (F) wherein genes within each set were altered with a P<0.05 threshold. P-values from the test of overlap significance are shown (bottom margin, Fisher's exact test). Histogram (G) presents the results of IDMF-decreased/MS-increased DEGs wherein the genes are ranked by FC: with respect to IGFBP5, CD74, UHRF1, BGN AND PPP1R13L the FDR<0.10). Histogram (H) presents the GO BP terms enriched among IDMF-decreased/MS-increased DEGs. The number of genes associated with each term is given in parentheses and example genes are listed within the figure.

Based on the results presented in FIG. 2, it is evident that genome-wide expression responses to DMF were positively correlated with those observed in MS patients (r=0.23), indicating that DMF elicited MS-like expression changes in astrocytes (scatterplot (A)). There was no significant overlap between DMF-increased genes and MS-decreased genes (P=1.00; gene set overlap (B)), and likewise, there was no significant overlap between DMF-decreased genes and MS-increased genes (P=0.99; gene set overlap (C)). Expression responses to DMF in cultured astrocytes, therefore, did not oppose, but in some respects replicated expression shifts observed in MS patient astrocytes.

In contrast, genome-wide expression responses to IDMF were negatively correlated with those observed in MS patients (scatterplot (D)), indicating that IDMF tended to oppose expression shifts seen with MS. 35.08% of protein-coding genes were increased by IDMF (FC>1.00) but decreased in MS patients (FC<1.00). This percentage was significantly larger-than-expected (P<0.05, Fisher's exact test. Genes increased by IDMF at a P<0.05 threshold did not significantly overlap with genes decreased by MS at the same threshold (P=0.26; gene set overlap (E)). However, genes decreased by IDMF at a P<0.05 threshold did overlap significantly with genes increased by MS at the same threshold (P=4.8e-09; gene set overlap (F)). Overall, 82 genes were decreased by IDMF (P<0.05) and increased in MS (P<0.05), including IGFBP5, CD74 and UHRF1 (histogram (G)). Many of the IDMF-decreased and MS-increased genes were associated with translation and targeting of proteins to the endoplasmic reticulum. For example, GO BP terms associated with such genes included SRP-dependent cotranslational protein targeting to membrane, establishment of protein localization to endoplasmic reticulum, and nuclear-transcribed mRNA catabolic process (nonsense-mediated decay). These results are indicative of a marked and surprising benefit from the use of IDMF in the treatment of MS.

### Example 4

In light of the recent studies regarding the neuroprotectant effect of DMF through NRF2 activation in the treatment of multiple sclerosis as shown by Gopal, S., et. al., “Evidence of activation of the Nrf2 pathway in multiple sclerosis patients treated with delayed-release dimethyl fumarate in the Phase 3 DEFINE and CONFIRM studies,” Mult Scler. 2017 December; 23(14):1875-1883. doi: 10.1177/1352458517690617. Epub 2017 Feb. 3 and Wang, Q., et. al. “Dimethyl Fumarate Protects Neural Stem/Progenitor Cells and Neurons from Oxidative Damage through Nrf2-ERK1/2 MAPK Pathway,” Int J Mol Sci, 2015 Jume 17:16(6):13885, a further study was conducted using the Cignal ARE Reporter Assay Kit to measure the transcriptional activity of NRF2 (NF-E2-related factor 2) transcription factor.

Stock solutions of DMF and IDMF were prepared in DMSO immediately before adding to cell cultures, and tested at serial aqueous dilutions of 1 μg/ml and 10 μg/ml and compared to a negative control comprising the diluted solvent. The experiments were performed using HEK293 cells (Sigma, St. Louis, Mo.; cat. #85120602) seeded at 50,000 per well in a 96 well white opaque-wall tissue culture plates. Cells were transfected by mixing Cignal Reporter Assay firefly/renilla luciferase constructs (cat. # CCS-5020L; Qiagen) with Attractene Transfection Reagent (Qiagen) in Opti-MEM (ThermoFisher Scientific, Waltham, Mass.) supplemented with 1% MEM Non-essential amino acid solution without L-glutamine (Sigma; cat. #M7145). After medium change test materials were added and their effect on NRF2 induction was quantified 24 h later with the Dual-Luciferase Reporter Assay System (cat. #E1960; Promega, Madison, Wis.).

The ARE (antioxidant response element) reporter is a mixture of a Nrf2-responsive luciferase construct encoding the firefly luciferase reporter gene under the control of a minimal (m)CMV promoter and tandem repeats of the ARE transcriptional response element, and a constitutively expressing Renilla element (40:1), which acts as an internal control for normalizing transfection efficiencies and monitoring cell viability. The number of response elements and the intervening sequence between these response elements has been experimentally optimized to maximize the signal to noise ratio. Signal quantification was obtained with ThermoFisher Scientific Luminoskan Ascent Microplate Luminometer. The results are presented in Table 4.

TABLE 4
DMF and IDMF on NRF2-mediated luciferase gene expression

| | | Fold Change v. | Standard error |
|---|---|---|---|
| Sample | Concentration | water control (1) | of mean |
| DMF | 1 μg/ml | 1.38 | ±0.03 |
| | 10 μg/ml | 1.81 | ±0.05 |
| IDMF | 1 μg/ml | 2.78 | ±0.28 |
| | 10 μg/ml | 4.61 | ±0.42 |

As evident from the results shown in Table 4, while DMF proved efficacious in enhancing NRF2-mediated luciferase expression, particularly at the higher concentration, IDMF provided a surprising and marked effect on NRF2-mediated luciferase expression at both concentrations. Furthermore, it is to be recalled that at these equal weights, DMF has approximately 0.5 moles of additional methyl fumarate for every mole of IDMF. Hence, at methyl fumarate molar equivalent ratios, i.e., 2 moles of DMF for every mole of IDMF, it is expected that the fold change difference between IDMF and DMF would have been significantly higher.

### Example 5—A1 v A2 Marker Activation of IDMF Vs DMF

DMF and IDMF (2.5 μM each) were applied to human fetal astrocytes for 24 hrs, with water employed as the negative control. The treated cells were stimulated with H₂O₂ (50 μM) for 20 min and the RNA extracted 1 day later and sequenced using the Illumina HiSeq 2000 with an average of 39.3 million reads per sample. Reads were mapped to the GHch38/hg38 genome. 13,968 protein-coding genes had detectable expression on average among the 6 samples. Differentially expressed genes (DEGs) were identified using negative binomial models with FDR threshold of <0.10 and fold change (FC) criteria (FC>1.30 or FC<0.77). Gene set enrichment testing was performed using public database annotations. Motif enrichment with respect to DEG up-stream regions was characterized using semiparametric generalized additive logistics models. The overlap between the identified DEGs and MS-associated genes, i.e., genes from MS patients and genes linked to inflammatory (A1) and ischemic (A2) astrogliosis. In-vitro fluorescence assays were used to evaluate effects of DMF and IDMF enzyme activity, and RTPCR was used to evaluate a subset of expression changes identified by RNA-seq. among the overlapping genes.

Among the overlapping genes was ICAM1, which is associated with the initiation and progression of inflammation as well as the targeting of inflammation in the central nervous system (CNS). IDMF was found to downregulate ICAM1, whereas, no similar activity was found with DMF indicative of an antagonism of inflammation in the CNS with IDMF, but not DMF. A similar result was observed with genes encoding cell cycle proteins. More importantly, 163, 198, and 85 genes associated with pan-reactive astrogliosis (A1+A2), A1-polarization, and A2-polarization, respectively were identified and assessed: the genes most strongly associated with each pattern included LCN2, C2ORF88, and VASH1, respectively. Although the effect size was small, there was a significant trend in which DMF tended to increase expression of genes linked to A1-polarization, i.e, neurotoxic or pro-inflammatory phenotype but, in contrast, decreased expression of genes linked to A2-polarization, i.e., neuroprotective or anti-inflammatory phenotype. In sharp contrast and supporting its efficacy in treatment of MS. IDMF tended to increase the expression of genes linked to A2 polarization while decreasing genes linked to pan-reactive gliosis: again, indicative of beneficial impact or repair.

### Example 6—IDMF v DMF Metabolism

IDMF and DMF were subjected to carboxylesterase hydrolysis. Specifically, a plurality of solutions of 1 mM each of IDMF and DMF were prepared by dissolving each in 2% DMSO+10% Ethylene Glycol in 20 mM HEPES pH 7.4. 1 mg/ml carboxylesterase 2 (CES2) was added to each solution and the solutions incubated at 37° C. for the time periods set forth in Table 5. A sample of each solution was removed at the time period and the reaction stopped with methanol and the samples evaluated and quantified for any remaining IDMF and DMF as well as, in the case of IDMF, IMMF, the metabolite intermediate, using an UV detector at 210 nm. Isosorbide was quantified using CAD. Mobile phase used for the study: 0.1% Formic acid in water; B: Methanol. Column: Luna C18 4.6 mm×100 mm, 5 u (Phenomenex). Flow rate: 1.0 ml/min.

The results are presented in Table 5. As seen in Table 5, all but about 17% of the DMF is hydrolyzed to MMF within thirty minutes and essentially all within 60 minutes. On the other hand, surprisingly, essentially all of the IDMF had hydrolyzed to MMF and IMMF within 15 minutes. Equally surprising is the finding that from that point forward, there was a slow hydrolysis of IMMF to MMF and isosorbide. As an orally administered pro-drug which undergoes carboxylesterase hydrolysis in the gastrointestinal tract, DMF rapidly and nearly completely converts to MMF. On the other hand, the surprising release pattern shown with IDMF provides for a quick infusion or release of MMF, but without an overloading of MMF as seen with DMF, and eliminates or greatly reduces the generation of methanol; both of which characteristics lessen the likelihood of GI distress and provide for improve tolerance in patients treated with the same. Additionally, since the IDMF is converted to IMMF first, the use of IDMF unexpectedly provides for MMF as well as a reservoir or supply of additional MMF in the form of IMMF, which slowly metabolizes to release the additional MMF: thereby providing for a longer release and continued elevated level of MMF in the body. Essentially, IDMF has the capability of providing the efficacy of multiple, spaced doses of DMF in a single administration as well as the ability to enable the use of lesser amounts to achieve a better result than with DMF.

TABLE 5

| Time | | | | Isosorbide |
|---|---|---|---|---|
| (minutes) | DMF (mM) | IDMF (mM) | IMMF (mM) | (mM) |
| 0 | 1 | 1.027 | 0 | 0 |
| 15 | — | 0.072 | 0.540 | 0 |
| 30 | 0.166 | ND | 0.367 | 0.176 |
| 60 | 0.08 | ND | 0.289 | 0.234 |
| 120 | 0.014 | ND | 0.191 | 0.455 |

Experimental II

A subsequent series of experiments was performed to demonstrate the effect and comparative effect of IDMF and DRF and MMF, the active metabolite of the foregoing as well as DMF, in various MS related pathways.

Astrocyte Cell Cultures

Experiments were performed using human fetal astrocytes derived from cerebral cortex (cat. no. 882A-05f; Cell Applications, Sant Diego, Calif.). Cells were grown to subconfluence in human astrocyte growth medium (cat. no. 820-500, Cell Applications). Stock solutions of MMF, DRF and IDMF were prepared in DMSO (50 mM) and subsequent dilutions were made using distilled water. The experiments were performed using 12 replicate cultures, each of which was assigned to one of 4 experimental treatments (CTL, n=3; MMF, n=3, DRF, n=3; IDMF, n=3). Test materials were added to subconfluent cultures at a concentration of 2.5 μM and cells were incubated for 24 hours.

RNA Processing

RNA was extracted following the incubation period using the RNAeasy Mini Plus kit (Qiagen, Hilden, Germany) and robotic Qiacube Connect station (Qiagen). Samples were shipped on dry ice to the Thermo Fisher Microarray Research Services Laboratory (Santa Clara, Calif.) for transcriptome analysis. RNA quality analysis was performed upon sample receipt using the NanoDrop Lite (Thermo Fisher Scientific, Waltham, Mass.). All samples had 260/280 nm absorbance ratios higher than 1.90, consistent with high-purity RNA.

### Example 7—MS-Associated Gene Impact with IDMF, MMF and DRF

In follow-up to the study of Example, 1 above, genes associated with multiple sclerosis were identified based upon seven established database sources: (1) the NHGRI-EBI genome-wide association (GWA) study catalogue, (2) the Disease Ontology (DO) database, (3) the DisGeNET (curated) database, (4) the DisGeNET (BeFree), (5) MalaCards, (6) the Medical Subject Heading (MESH) database and the KEGG Database. The impact on gene expression of IDMF, MMF and DRF on the key overlapping MS-associated genes was assessed. The genes for this study as well as the impact thereon is presented in the table in FIG. 4. As seen, while there is considerable overlap in performance, there are marked differences as well: this is particularly so with respect to the marked and surprising downregulation of many MS-associated genes by IDMF (shown in dark blue in the lower portion of the table). Equally surprising is the significant upregulation of many MS-associated genes by IDMF, which were downregulated by MMF and/or DRF. FIGS. 5(A), 5(B) and 5(C) show the effect of MMF, DRF and IDMF, respectively, on those MS-associated genes most significantly affected the respective fumarate. These results demonstrate different impact and mechanisms involved in the treatment of MS by the different fumaric acid esters.

### Example 8—NRF Pathways

The mechanism of action of DMF is often understood in terms of its activation of the NFR2 pathways, specifically the NRF2-dependent and NRF2-independent pathways, with the former postulated to mediate a neuroprotective effect on CNS cell types and the latter affecting inhibition of neuroinflammation and with modulation of peripheral immune cells. Assays were performed to assess the impact of IDMF, DRF and MMF on these same pathways in an effort to assess the cytoprotective and anti-inflammatory/antioxidant effect of these fumarates. Plots of the impact of MMF and DRF on the NRF2 target genes are shown in an overlay relationship with bar charts of the impact of IDMF on the same genes are presented in FIGS. 6(A) and 6(B): the former presenting the NRF2-increase genes and the latter presenting the NRF2-decreased genes. While the correspondence of responsiveness was fairly similar amongst the three fumarates, surprisingly, IDMF had a much more pronounced effect in most cases, indicative of a higher level of performance and benefit as opposed to the other two, including the key active metabolite of DMF, MMF. The specific data for four of the key NFR2 target genes associated with antioxidant and anti-inflammatory effects is presented in FIGS. 7(A) thru 7(D). Most notable is the marked increase in expression of heme oxygenase-1 (HMOX1); an important gene in mediation of antioxidant effects. Accordingly, these results indicate that IDMF will have an improved antioxidant and anti-inflammatory benefit in MS treatment as compared to DMF and DRF.

### Example 9—NF-kB Pathways

A similar study was undertaken focusing on the NF-kB pathways, pathways inhibited by DMF. Plots of the impact of MMF and DRF on the NF-kB (RELA) target genes are shown in an overlay relationship with bar charts of the impact of IDMF on the same genes are presented in FIGS. 8(A) and 8(B): the former presenting the RELA-activated genes and the latter presenting the RELA-suppressed genes. As seen in FIGS. 8(A) and 8(B), while MMF and IDMF were fairly comparable in their downregulation of various NF-kB genes, DRF oftentimes did not demonstrate such downregulation. More importantly, a number of prominent NF-kB target genes, particularly TNFAIP3, NFKBIA and ICAM1, were down-regulated only by IDMF. Both TNFAIP3 and NFKBIA encode inhibitory proteins that suppress the NF-KB pathway. Specifically, TNFAIP3 encodes a deubiquitinating enzyme previously linked to MS by genetic association studies: a TNFAIP3 protein abundance is increased in white matter and cortical lesion astrocytes from MS patient brains. Similarly, NFKBIA (IκBα) encodes an NF-KB inhibitor for which promoter polymorphisms have been associated with MS and abundance of this protein is prominent in macrophage nuclei of MS plaques undergoing active demyelination. ICAM1 encodes an endothelial adhesion molecule that promotes leucocyte extravasation and is associated with the initiation and progression of inflammation as well as the targeting of inflammation in the central nervous system (CNS). FIGS. 9(A), 9(B) and 9(C) show the specific data for these three genes. Clearly, while MMF and DRF had effectively no effect on these genes, IDMF provided a marked downregulation. These results indicate that IDMF will have a marked beneficial effect in inhibiting the progenesis of MS and inflammation associated therewith as well as in the repair of damage caused by MS.

### Example 10—IFR Motif Enrichment

Genetic variants of IFR has been associated with MS and some MS treatments such as glatiramer acetate block cytokine-stimulated IRF1 up-regulation. An assessment was undertaken to assess the relative impact of fumarate esters, namely MMF, DRF and IDMF on IFR motif enrichment. FIGS. 10(A) through 10((F) demonstrate the effect found on the expression of six key IFRs. These findings demonstrate that IDMF manifested a marked downregulation of IRF1 expression, indicating a strong attenuation in cytotoxicity in oligodendroglia progenitor cells and limitation on pyroptosis in oligodendrocytes, hence, anti-inflammatory behavior. In following, this is indicative of IDMF having anti-inflammatory effects at multiple CNS and peripheral immune system sites.

### Example 11—Comparative RT-PCR Evaluation

The expression of selected genes was further evaluated using RT-PCR. cDNA was prepared using the AzuraQuant cDNA kit (Azura Genomics, Raynham, Mass.) and PCR reactions were performed using the BioRad iCycler iQ Detection System with Fast Green qPCR Master Mix—Fluor (Azura Genomics). PCR primers were purchased from Realtimeprimers (Elkins Park, Pa.). The ΔΔCt method [Livak K. J. and Schmittgen T. D. Analysis of relative gene expression data using real-time quantitative PCR and the 2(−Delta Delta C(T)) Method. Methods (San Diego, Calif.) 25(4):402-408, 2001]. was used to estimate fold changes with cycle threshold values normalized based upon the expression of glyceraldehyde-3-phosphate dehydrogenase (GAPDH). The specific genes tested and the results attained therewith are summarized in FIG. 11(A) through 11(H).

TNF is a known inducer of NF-kB, as shown in FIG. 11(A), the combination of IDMF had a marked effect on TNF activation of NF-kB, indicative of a possible synergistic effect in addressing inflammation association with MS. As shown in FIG. 11(B), IDMF had a marked effect on OSGIN1 expression, enhancing expression as compared to the suppression with MMF and DRF. OSGIN1 is associated with cytoprotective effects and its enhanced expression is expected to inhibit MS pathogenesis as well as reduce oxidative stress and inflammation in cells associated with MS.

Most significantly, as shown in FIGS. 11(C) through 11(H), IDMF had a marked suppression of expression of the identified, key MS-associated genes as compared to the lack or marginally enhanced expression found with MMF and DRF. In MS patients, IL-6 within the central nervous system (CNS) is localized to the astrocytes and concentrated in demyelinated region, indicative of its activity in the pathogenesis of MS and its attendant inflammation. ICAM1 is similarly associated with the initiation and progression of inflammation as well as the targeting of inflammation in the central nervous system (CNS). Although still under investigation, inhibition of MALT1 protease activity has been shown to be protective of MS damage in mouse models. TNFAIP3 is associated with the progression and pathogenesis of a variety of autoimmune diseases, especially MS. As noted above, IRF1 is associated with cytotoxicity in oligodendroglia progenitor cells and pyroptosis in oligodendrocytes, both detrimental manifestations of MS. Finally, CXCL8 is a mediator of the immune response and inflammation. Given the impact each of these genes has on the pathogenesis of MS and it symptoms, especially inflammation, it is clear that an effective treatment capable of inhibiting their expression will slow down and, perhaps, provide some measure of repair with respect to MS. Surprisingly, as shown in FIGS. 11(C) through 11(H), IDMF had a marked suppression effect, thus indicative of a protective, if not reparative, effect, as compared to the negligible effect of MMF and DRF. Hence, IDMF appears to unexpected and superior performance as an effective agent in the treatment of MS.

### Example 12—IDMF v DRF Metabolism

Similar to Example 6 above, the hydrolysis of IDMF was compared to that of DRF. Specifically, a plurality of solutions of 1 mM each of IDMF and DRF were prepared by dissolving each in 2% DMSO+10% Ethylene Glycol in 20 mM HEPES pH 7.4. 20 μl of 1 mg/ml carboxylesterase 2 (CES2) was added to each solution and the solutions incubated at 37° C. for the time periods set forth in Table 5. A sample of each solution was removed at the time period and the reaction stopped with methanol and the samples evaluated and quantified for any remaining IDMF and DMF as well as, in the case of IDMF, IMMF formation and, in the case of DRF, N-(2-hydroxyetheyl) succinimide (HES). Although tolerable for normal patients, HES is undesirable and detrimental for those individuals have kidney and/or liver issues

The results are presented in Table 6. As seen in Table 6, DRF had a very slow, but steady, conversion to MMF with a reciprocal conversion and buildup of HES. As with the prior showing, IDMF had an initial, quick generation of MMF, evident of the conversion of IDMF to IMMF and MMF, followed by a longer term, slower generation of MMF indicative of the hydrolysis of IMMF to MMF and isosorbide.

TABLE 6

| Time | | | | |
|---|---|---|---|---|
| (minutes) | DRF (mM) | HES (mM) | IDMF (mM) | IMMF (mM) |
| 0 | 1 | ND | 1.086 | ND |
| 15 | .652 | 0.075 | 0.113 | 1.181 |
| 30 | 0.482 | 0.103 | ND | 1.585 |
| 60 | 0.273 | 0.127 | ND | 1.713 |
| 120 | 0.08 | 0.558 | ND | 1.721 |

Without further elaboration, it is believed that one skilled in the art, using the preceding description, can utilize the present invention to its fullest extent. Furthermore, while the present invention has been described with respect to aforementioned specific embodiments and examples, it should be appreciated that other embodiments, changes and modifications utilizing the concept of the present invention are possible, and within the skill of one in the art, without departing from the spirit and scope of the invention. The preceding preferred specific embodiments are, therefore, to be construed as merely illustrative, and not limitative of the remainder of the disclosure in any way whatsoever.

We claim:

1. A method of treating multiple sclerosis or the symptoms thereof said method comprising administering to the patient suffering therefrom a therapeutically effective amount of a compound according to Formula (I): 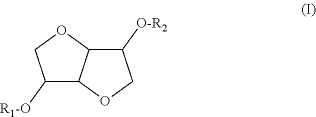
in which R₁ and R₂ are both —C(O)CH═CH—C(O)OMe or one of R₁ and R₂ is H and the other —C(O)CH═CH—C(O)OMe, wherein Me is methyl.

2. The method of claim 1 wherein the compound is of the formula (I)(a)

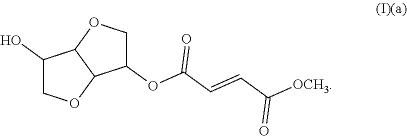 [SMILES: COC(=O)/C=C/C(=O)OC1COC2C(O)COC12]

3. The method of claim 1 wherein the compound is of the formula (I)(b)

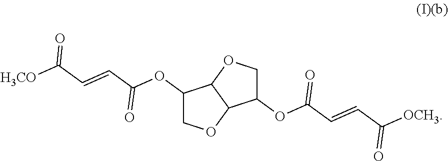 [SMILES: COC(=O)/C=C/C(=O)OC1COC2C(OC(=O)/C=C/C(=O)OC)COC12]

4. The method of claim 1 wherein the compound is isosorbide di-(methylfumarate).

5. The method of claim 1 wherein the compound is administered systemically.

6. The method of claim 1 wherein the compound is administered locally to one or more areas of a patient where manifestation of the symptoms of MS is found.

7. The method of claim 1 wherein the compound is also administered orally.

8. A method of treating multiple sclerosis or the symptoms thereof said method comprising administering to the patient suffering therefrom a therapeutically effective amount of composition comprising at least one compound according to Formula (I): 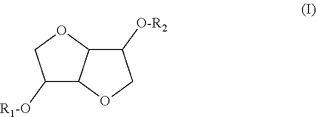
in which R₁ and R₂ are both —C(O)CH═CH—C(O)OMe or one of R₁ and R₂ is H and the other —C(O)CH═CH—C(O)OMe, wherein Me is methyl and a pharmaceutically acceptable vehicle.

9. The method of claim 8 wherein the compound is of the formula (I)(a)

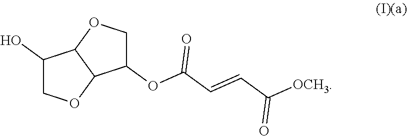 [SMILES: COC(=O)/C=C/C(=O)OC1COC2C(O)COC12]

10. The method of claim 8 wherein the compound is of the formula (I)(b)

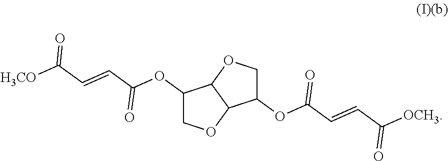 [SMILES: COC(=O)/C=C/C(=O)OC1COC2C(OC(=O)/C=C/C(=O)OC)COC12]

11. The method of claim 8 wherein the compound is isosorbide di-(methylfumarate).

12. The method of claim 8 wherein the at least one compound of formula (I) comprises a combination of isosorbide di-(methylfumarate) and isosorbide mono-(methyfumarate).

13. The method of claim 8 wherein the composition further comprises at least one other pharmaceutical active.

14. The method of claim 13 wherein the other pharmaceutical active is one that reduces, ameliorates, inhibits or otherwise addresses or treats MS symptoms and/or conditions associated with MS and its concurrent diseases/maladies.

15. The method of claim 13 wherein the other pharmaceutical active is selected from Fingolimod or Teriflunomide or a combination thereof.

16. The method of claim 8 wherein the composition is administered systemically.

17. The method of claim 8 wherein the composition is administered locally to one or more areas of a patient where manifestation of the symptoms of MS is found.

18. The method of claim 8 wherein the composition is administered orally.

19. A composition for use in the treatment of multiple sclerosis or the symptoms thereof said composition comprising administering to the patient suffering therefrom a therapeutically effective amount of composition comprising at least one compound according to Formula (I): 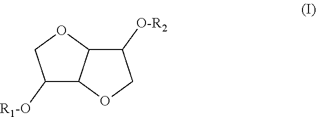
in which R₁ and R₂ are both —C(O)CH═CH—C(O)OMe or one of R₁ and R₂ is H and the other —C(O)CH═CH—C(O)OMe, wherein Me is methyl, and at least one other pharmaceutical active that reduces, ameliorates, inhibits or otherwise addresses or treats MS symptoms and/or conditions associated with MS and its concurrent diseases/maladies, and a pharmaceutically acceptable vehicle.

20. The composition of claim 19 wherein the other pharmaceutical active is selected from Fingolimod or Teriflunomide or a combination thereof.

21. The composition of claim 19 wherein the compound is of the formula (I)(a)

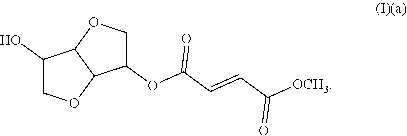 [SMILES: COC(=O)/C=C/C(=O)OC1COC2C(O)COC12]

22. The composition of claim 19 wherein the compound is of the formula (I)(b)

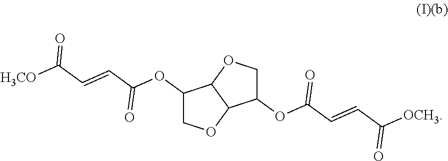 [SMILES: COC(=O)/C=C/C(=O)OC1COC2C(OC(=O)/C=C/C(=O)OC)COC12]

23. The composition of claim 19 wherein the compound is isosorbide di-(methylfumarate).
